(19) United States
(12) Patent Application Publication
(10) Pub. No.: US 2007/0232556 A1
(43) Pub. Date: Oct. 4, 2007

(54) Methods and compositions for the treatment of neurological diseases and disorders

(76) Inventors: Thomas J. Montine, Bellevue, WA (US); Feng-Shiun Shie, Hsin Chu City (TW)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/396,212

(22) Filed: Mar. 31, 2006

(51) Int. Cl. A61K 48/00; A61K 38/17; A61K 31/557; A61K 39/395

(52) U.S. Cl. 514/44; 514/2; 514/573; 424/143.1

(57) ABSTRACT

Methods and compositions are provided for preventing or treating neurodegenerative disease and other related diseases and disorders in a mammalian subject. The method provides administering to the mammalian subject a compound capable of inhibiting a PGE2 receptor, e.g., subtype EP2, wherein the compound is administered in an amount effective to treat the neurodegenerative disease or to prevent its occurrence or recurrence. A method for identifying compounds capable of inhibiting neurodegenerative disease is also provided.

### GOVERNMENT SUPPORT

This invention was made with Government support under NIH grant R01 AG 24011. The Government has certain rights to this invention.

### FIELD

The invention relates generally to the treatment of neurological diseases and disorders.

### BACKGROUND

Microglia are thought to play an important role in pathogenesis of several neurodegenerative diseases where they become activated, displaying both innate immune response and increased phagocytic activity. A robust innate immune response occurs in association with Aβ-containing plaques in brain of patients with Alzheimer disease (AD) that is characterized by activation of complement, increased secretion of several cytokines and chemokines, increased production of reactive oxygen and nitrogen species, and increased production of prostaglandin (PG) E2 (Cooper et al., Neurobiol Aging 21: 451-453, 2000; El Khoury et al., Nature 382: 716-719, 1996; Husemann et al., J Neuroimmunol 114: 142-150, 2001; Lue et al., Glia 35: 72-79, 2001), some combination of which can be neurotoxic. Indeed, Aβ is one of handful of endogenous ligands known to activate innate immunity and thereby is proposed to contribute to AD initiation and early progression (D'Andrea et al., Neurobiol Aging 25: 675-683, 2004; McGeer et al., Neurobiol Aging 22: 799-809, 2001). Consistent with this hypothesis, several large observational studies have repeatedly observed that long-term use of nonsteroidal anti-inflammatory drugs (NSAIDs) is associated with a decreased incidence of AD (Szekely et al., Neuroepidemiology 23: 159-169, 2003; McGeer et al., Neurology 47: 425-432, 1996) although other mechanisms of action for some NSAIDs have been proposed (Weggen et al., Nature 414: 212-216, 2001).

PGE₂, a product derived from arachidonic acid by cyclooxygenase (COX) and specific synthases, is a potent autocrine and paracrine factor that is distinct from other eicosanoid products of COX because of multiple G-protein coupled receptor subtypes, EP1, EP2, EP3, and EP4 that are linked to functionally antagonistic second messenger systems (Narumiya et al., Physiol Rev 79: 1193-1226, 1999). All EP receptor subtypes are expressed on varying cells in brain; microglia express EP1 and EP2, and perhaps EP3 when activated (Caggiano et al., J Neurochem 72: 565-575, 1999). Because of this complexity, PGE₂ has been conflictingly reported to mediate neurotoxicity and to be neuroprotective, as well as to enhance and suppress macrophage phagocytosis (Aronoff et al., J Immunol 173: 559-565, 2004; Zhang et al., J Neurochem 76: 855-864, 2001; Petrova et al., J Biol Chem 274: 28823-28827, 1999).

Recent results from large observational studies and clinical trials in individuals at risk for developing AD point specifically to products of COX and lipid peroxidation as potential targets for reducing the incidence of AD (Anthony et al., J. Neurology 54: 2066-2071, 2000; Stewart et al., Neurology 48: 626-623, 1997; in 't Veld et al., N Engl J Med 345: 1515-1521, 2001; Andersen et al., Neurology 45: 1441-5, 1995; Stewart et al., Neurology 48: 626-632, 1997) however, in both cases treatment trials in patients with established AD have been disappointing.

Most experiments testing hypotheses about PGE2 in AD have focused on its role in the activation of glia that then release effectors of oxidative damage and neurodegeneration. These effectors include interleukin (IL)-1β from microglia, IL-6 from astrocytes, and nitric oxide (NO) and superoxide anion from both cell types; the latter two are thought to be major causes of oxidative damage (reviewed in Hardy and Selkoe, Science 297: 353-356, 2002). Several laboratories have indicated potent autocrine and paracrine roles for PGE2 in the activation of innate immunity in brain; however the results have been conflicting with some concluding that PGE2 contributes to neurodegeneration, while others concluded that PGE2 has neuroprotective activity. See Kelley et al., Am J Pathol 155: 995-1004, 1999; Sanzgiri et al., J Neurobiol 41: 221-229, 1999; Caggiano and Kraig, J Neurochem 70: 2357-2368, 1998; Caggiano and Kraig, J Neurochem 72: 565-575, 1999; Aloisi et al., J Neurosci Res 56: 571-580, 1999; Levi et al., Biochimie 80: 899-904, 1998; Minghetti et al., Glia 19: 152-160, 1997; Minghetti et al., Eur J Neurosci 9: 934-940, 1997; Minghetti et al., J Neuroimmunol 82: 31-39, 1998; Minghetti et al., J Neuroimmunol 82: 31-39, 1998). Similar contradictory data exists concerning the role of PGE2 in models of ischemia, seizure, and excitotoxicity (see Hewett et al., J Pharm Exp Ther 293: 417-425, 2000; Cazevielle et al., Neurochem Int 24: 395-398, 1994; Akaike, A., Brain Res 663: 237-243, 1994; Bezzi et al., Nature 391: 281-285, 1998; Sanzgiri et al., J Neurobiol 41: 221-229, 1999; Drachman et al., Ann Neurol 48: 792-795, 2000; Paoletti et al., Exp Neurol 149: 349-355, 1998; Thornhill and Smith, Brain Res 784: 48-56, 1998). To summarize these findings, in vivo experiments that have blocked COX activity with NSAIDs suggest that PGE2 promotes oxidative damage and neurodegeneration, while experiments using cultured neurons or glia exposed to exogenous PGE2 imply that PGE2 is neuroprotective.

Therefore there exists a need to resolve the apparent paradox of PGE2 neurodegenerative and neuroprotective activity and to guide the use and timing of agents to suppress PGE2 production in patients with neurodegenerative diseases and disorders.

### BRIEF SUMMARY

The present invention provides a method for preventing or treating a neurological disease in a mammalian subject comprising administering to the mammalian subject a compound capable of inhibiting prostaglandin E2 (PGE2) receptor, wherein the compound is administered in an amount effective to treat the neurological disease or to prevent its occurrence or recurrence. The target of the inhibitor is a PGE2 receptor subtype, including but not limited to EP2. The inhibitor can be a small chemical compound, dominant-negative molecule, siRNA, ribozyme, antisense, or antibody. In some aspects, the neurological disease is Alzheimer's disease or Parkinson's disease. In some aspects, the compound enhances microglial phagocytosis of pathogenic proteins associated with neurological diseases and disorders. In some such aspects, the pathogenic proteins are Aβ proteins associated with the initiation and progression of Alzheimer's disease. In other such aspects, the pathogenic proteins are synuclein proteins, e.g., α-synuclein proteins associated with the initiation and progression of Parkinson's disease. In some aspects, the compound promotes β amyloid (Aβ) clearance.

In another aspect, the invention provides an in vitro method of screening for an inhibitor of PGE2 receptor biological activity comprising: contacting a cell with a test compound; and detecting an increase or a decrease in activation, thereby identifying the test compound as an inhibitor of EP receptor biological activity. In some aspects, PGE2 receptor is EP2. In other aspects, the cell is an astrocyte, microglia or neuron. In other aspects, compounds are identified by the in vitro method.

In another aspect, the invention provides a method for identifying a compound capable of inhibiting PGE2 receptor signaling comprising: contacting a test compound with a cell-based assay system comprising a cell expressing a PGE2 receptor capable of signaling responsiveness to PGE2 receptor signaling, and detecting an effect of the test compound on PGE2 receptor signaling in the assay system as an increase or a decrease in second messenger signaling or physiologic outcome, effectiveness of the test compound in the assay being indicative of the inhibition EP receptor signaling. In some aspects, the physiologic outcome is an increase or decrease in phagocytosis. In other aspects, the test compound is a small chemical molecule, interfering RNA, short hairpin RNA, ribozyme, antisense oligonucleotide, protein inhibitor, monoclonal antibody, a polyclonal antibody, a peptide, or a nucleic acid. In some aspects, the PGE2 receptor is subtype EP2. In other aspects, compounds are identified by the in vitro method. In some aspects, the compound is an EP2 receptor subtype antagonist.

In another aspect, compounds are identified by the in vitro method. Some compounds alter the biological activity of the EP2 receptor subtype. Some such compounds are EP2 receptor subtype antagonists.

In another aspect, the invention provides a pharmaceutical composition comprising a PGE2 inhibitor for the treatment of a neurological disease or disorder in a mammalian subject. In some aspects, the PGE2 inhibitor inhibits PGE2 receptor subtype EP2. In other aspects, the inhibitor is a small chemical compound, interfering RNA, dominant-negative molecule, short hairpin RNA, ribozyme, antisense oligonucleotide, or protein inhibitor. In some aspects, the neurological disease is Alzheimer's disease or Parkinson's disease. In other aspects, the composition promotes Aβ clearance from the brain. In some aspects, the composition promotes synuclein clearance from the brain. In some such compositions, the synuclein is α-synuclein.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1. Enhanced phagocytosis of Aβ from senile plaques in human hippocampus by EP2^{-/-} microglia ex vivo. AD brain sections with primary microglial cultures from wt or EP2^{-/-} mouse cerebrum were fixed and subjected to immunofluorescent labeling. Representative confocal images containing similar number of microglia are presented. FIG. 1A shows tissue with Aβ-IR material (green) in the pattern of senile plaques, wt microglia (red), and nuclei (blue); the density and pattern of Aβ-IR plaque material in tissue incubated with wt microglia was no different from tissue sections incubated without microglia (not shown). Consecutive tissue section incubated with EP2^{-/-} microglia showed marked disappearance of Aβ-IR material (FIG. 1B). Pseudopodia engulfing Aβ-IR plaque material were also seen in EP2^{-/-} microglia (arrow in inset of FIG. 1B). At a higher magnification, EP2^{-/-} activated microglia contained Aβ-IR material (FIG. 1D) inside phagocytic vacuoles as indicated by intracellular compartments IR for the integrin marker CD11b (FIG. 1E). In addition, DNA fragments were also seen within these vacuoles (FIG. 1F). FIG. 1C is a merge of FIGS. 1D, 1E, and 1F. Magnifications: A-B, 250×; C-F, 1000×.

FIG. 2. Microglial activation and reduction of Aβ1-40 and Aβ1-42 in human hippocampus by EP2^{-/-} microglia ex vivo. AD brain sections with primary microglial cultures from wt or EP2^{-/-} mouse cerebrum were subjected to urea extraction followed by Western blot analysis (representative blot in FIG. 2A). There was no difference in band density for Aβ1-40 or Aβ1-42 when tissue sections incubated without microglia were compared to sections incubated with wt microglia (not shown). Aβ1-40 was reduced 44+5% (n=6, p<0.05) and Aβ1-42 was reduced 43%+4% (n=6, p<0.01) in human hippocampus incubated with EP2^{-/-} microglia compared to tissue incubated with wt microglia (FIG. 2B). Intensity of CD11b immunoreactivity per microglial cell was evaluated by confocal images from wt (FIG. 2C) and EP2^{-/-} (FIG. 2D) on human hippocampal slices and quantified (FIG. 2E); fluorescence intensities (in arbitrary units) in wt microglia and EP2^{-/-} microglia were 23+2 (n=5) and 110+7 (n=10, p<0.01), respectively. Magnification: C-D, 250×.

FIG. 3. Microglial phagocytosis of aggregated fluorescein-labeled Aβ1-42. Phagocytosis of Aβ1-42 was quantified by flow cytometry; some microglia were cultivated in chamber slides for fluorescent analysis. Representative fluorescence images (200×, green is fluorescein label, nuclei are blue) of primary microglia cultures from chamber slides with wt mice (A) or EP2^{-/-} mice (B) showed an increased proportion of cells with enhanced fluorescence intensity in EP2^{-/-} microglia compared to wt. Representative flow cytometric scatter plot (C) showed that granularity and size were not different between wt (blue) and EP2^{-/-} (red) microglia. Flow cytometric histogram in log scale (D) showed a shift toward higher fluorescent intensity in EP2^{-/-} microglia compared to wt. Defining high Aβ1-42 fluorescent intensity as that greater than the peak value for EP2^{-/-} microglia, we determined the percentage of the tested cells with high fluorescent intensity between wt and EP2^{-/-} microglia; representative histograms are shown in E and F.

FIG. 4. Signaling pathways for enhanced Aβ1-42 phagocytosis in EP2^{-/-} microglia. Fluorescein-labeled Aβ1-42 phagocytosis was quantified by flow cytometry as above. Data from wt microglia are in solid bars while data from EP2^{-/-} microglia are in striped bars. Basal Aβ1-42 uptake in EP2^{-/-} microglia (n=16) was 263+13% of wt microglia (n=7; t-test had +P<0.0001). Drugs were used to test the involvement of specific signaling pathways. ANOVA for 5 treatment groups of wt microglia had P<0.0001 with Bonferroni-corrected posttests having P<0.001 for untreated vs. BIM but P>0.05 for all other comparisons to untreated wt microglia. ANOVA for 5 treatment groups of EP2^{-/-} microglia had P<0.0001 with Bonferroni-corrected posttests having P<0.001 for untreated vs. BIM but P>0.05 for all other comparisons to untreated EP2^{-/-} microglia.

FIG. 5. Morphological changes in primary neurons by Aβ toxicity. To test whether Aβ-activated EP2^{-/-} microglia had enhanced paracrine neurotoxicity, co-cultures of primary wt neurons (DIV 6) and microglia (DIV 14) from either wt or EP2^{-/-} mice were used for assessment of morphological changes in neurons using MAP-2 antibody. Results showed that neurons alone without treatment (A) displayed extensively branched dendrites, while exposure of neurons alone to 12 μM of aggregated Aβ1-42 (B) was associated with decreased dendrite length as assessed by MAP-2 IR; even greater Aβ1-42-initiated dendritic atrophy was observed in cultures that combined neurons and wt microglia (C). Strikingly, cultures that contained neurons and EP2^{-/-} microglia (D) showed no evidence of neurotoxicity by morphologic assessment and were comparable to what was observed with neurons alone without Aβ1-42 exposure. Magnification: 200×.

### DETAILED DESCRIPTION

1. General Introduction and Overview

A method for preventing or treating a neurological disease in a mammalian subject is provided comprising administering to the mammalian subject a compound capable of modulating prostaglandin E2 (PGE2) receptor, wherein the compound is administered in an amount effective to reduce or eliminate the neurological disease or to prevent its occurrence or recurrence. The target of the inhibitor is a PGE2 receptor subtype, including but not limited to, the EP2 receptor subtype. The inhibitor can be, for example, a small chemical compound, siRNA, ribozyme, antisense, or antibody.

It is to be understood that this invention is not limited to particular methods, reagents, compounds compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Neurodegenerative diseases or disorders” refers to hereditary and sporadic conditions which are characterized by progressive nervous system dysfunction. These disorders are often associated with atrophy of the affected central or peripheral nervous system structures. Alzheimer's disease, Parkinson's disease, Creutzfeldt-Jakob, as well as multiple sclerosis, are due to neuronal degeneration in the central nervous system (discussed in more detail below).

“Amyloidosis” or “amyloid disease” refers to a pathological condition characterized by the presence of amyloid fibers. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra. The term “amyloid-β diseases” includes those diseases, conditions, pathologies, and other abnormalities of the structure or function of the brain, including components thereof, in which the causative agent is amyloid. The area of the brain affected in an amyloid-β disease can be the stroma including the vasculature or the parenchyma including functional or anatomical regions, or neurons themselves. A subject need not have received a definitive diagnosis of a specifically recognized amyloid-β disease.

Local deposition of amyloid is common in the brain, particularly in elderly individuals. The most frequent type of amyloid in the brain is composed primarily of Aβ peptide fibrils, resulting in dementia associated with sporadic (non-hereditary) Alzheimer's disease. In fact, the incidence of sporadic Alzheimer's disease greatly exceeds forms shown to be hereditary. Nevertheless, fibril peptides forming plaques are very similar in both types.

“Alzheimer's disease”, also referred to as “AD”, is a syndrome with several monogenetic autosomal dominant causes, each related to metabolism of the amyloid precursor protein (APP) and its cleavage to form amyloid β (Aβ) peptides. However, the much more common form(s) of AD does not have a single genetic cause. This last group, referred to as sporadic AD, likely derives significant input from the intertwined processes of aging, inherited susceptibilities such as the ε4 allele of the apolipoprotein (apo) E gene (APOE4), and environmental factors (St. George-Hyslop, G., Genetics of Alzheimer's disease, in Markesbery WR (ed): Neuropathology of Dementing Disorders, 106-120, 1998). A dominant hypothesis is that all forms of AD share in common increased production or accumulation of Aβ peptides, especially Aβ₄₂. There is also abundant pathological and biochemical data indicating that activation of innate immunity, at least in part by aggregated Aβ peptides, is a central feature of sporadic AD and is a likely cause of at least some of the oxidative damage that characterizes AD (reviewed in Hardy and Selkoe, Science 297: 353-356, 2002). Specific receptor-mediated events that lead from activation of innate immunity to oxidative damage are discussed herein.

Amyloid-β peptide (Aβ) is a 39-43 amino acid peptide derived by proteolysis from a large protein known as Beta Amyloid Precursor Protein (“βAPP”). Mutations in βAPP result in familial forms of Alzheimer's disease, Down's syndrome, cerebral amyloid angiopathy, and senile dementia, characterized by cerebral deposition of plaques composed of Aβ fibrils and other components. Known mutations in APP associated with Alzheimer's disease occur proximate to the cleavage sites of βor γ-secretase, or within Aβ. For example, position 717 is proximate to the site of gamma-secretase cleavage of APP in its processing to Aβ and positions 670/671 are proximate to the site of β-secretase cleavage. Mutations at any of these residues can result in Alzheimer's disease, presumably by causing an increase in the amount of the 42/43 amino acid form of Aβgenerated from APP. The familial form of Alzheimer's disease represents only 10% of the subject population. Most occurrences of Alzheimer's disease are sporadic cases where APP and Aβ do not possess any mutation.

The structure and sequence of Aβ peptides of various lengths are well known in the art. Such peptides can be made according to methods known in the art, or extracted from the brain according to known methods (e.g., Glenner and Wong, Biochem. Biophys. Res. Comm. 129: 885-90, 1984; Glenner and Wong, Biochem. Biophys. Res. Comm. 122: 1131-35, 1984). In addition, various forms of the peptides are commercially available.

As used herein, the terms “β-amyloid,” “amyloid-β,” and the like refer to amyloid β proteins or peptides, amyloid β precursor proteins or peptides, intermediates, and modifications and fragments thereof, unless otherwise specifically indicated. In particular, “Aβ” refers to any peptide produced by proteolytic processing of the APP gene product, especially peptides which are associated with amyloid pathologies, including Aβ1-39, Aβ1-40, Aβ1-41, Aβ1-42, and Aβ1-43. For convenience of nomenclature, “Aβ₁₋₄₂” can be referred to herein as “Aβ(1-42)” or simply as “Aβ42” or “Aβ₄₂” (and likewise for any other amyloid peptides discussed herein). As used herein, the termsβ “amyloid,” “amyloid-β,” and “Aβ” are synonymous. Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque). See, e.g., Lambert et al., Proc. Nat'l Acad. Sci. 95: 6448-53, 1998.

According to certain aspects of the invention, amyloid-β is a peptide having 39-43 amino-acids, or amyloid-β is an amyloidogenic peptide produced from β APP. The amyloid-β diseases that are the subject of the present invention include age-related cognitive decline, early Alzheimer's disease as seen in Mild Cognitive Impairment (“MCI”), vascular dementia, or Alzheimer's disease (“AD”), which can be sporadic (non-hereditary) Alzheimer's disease or familial (hereditary) Alzheimer's disease. The amyloid-β disease can also be cerebral amyloid angiopathy (“CAA”) or hereditary cerebral hemorrhage. The amyloid-β disease can be senile dementia, Down's syndrome, inclusion body myositis (“IBM”), or age-related macular degeneration (“ARMD”).

“Parkinson's disease”, also referred to as “PD”, is a chronic disease of the central nervous system. Parkinson's disease is characterized by the presence of Lewy bodies and the loss of dopamine-producing neurons in substantia nigra that controls muscle movement. The Lewy body is an abnormal structure found in certain areas of the brain. It contains a protein called α-synuclein, which plays the central role in Parkinson's disease and other diseases involving Lewy bodies, such as dementia with Lewy bodies, multiple system atrophy, and Hallervorden-Spatz disease (Jellinger, Mov Disord. 18 (Suppl 6): S2-12, 2003).

“Synucleins” are small, soluble proteins expressed primarily in neural tissue and in certain tumors (for a review, see Lavedan, Genome Res. 8: 871-80, 1998; see also George, Genome Biol. 3: 3002.1-3002.6, 2002). The family includes three known proteins: alpha-synuclein (α-synuclein), beta-synuclein (β-synuclein), and gamma-synuclein (γ-synuclein). All synucleins have in common a highly conserved alpha-helical lipid-binding motif with similarity to the class-A2 lipid-binding domains of the exchangeable apolipoproteins (Perrin et al., J. Biol. Chem. 275: 34393-8, 2000).

Synuclein family members are not found outside vertebrates, although they have some conserved structural similarity with plant “late-embryo-abundant” proteins. The alpha- and beta-synuclein proteins are found primarily in brain tissue, where they are seen mainly in presynaptic terminals (Iwai et al., Neuron 14: 467-75, 1995; Nakajo et al., Brain Res Mol Brain Res. 27: 81-6, 1994). The gamma-synuclein protein is found primarily in the peripheral nervous system and retina, but its expression in breast tumors is a marker for tumor progression (Ji et al., Cancer Res. 57: 759-64, 1997). Some data suggest a role in the regulation of membrane stability and/or turnover. Mutations in alpha-synuclein are associated with rare familial cases of early-onset Parkinson's disease, and the protein accumulates abnormally in Parkinson's disease, Alzheimer's disease, and several other neurodegenerative illnesses (Goedert, Nat Rev. Neurosci, 2: 492-501, 2001).

“α-Synuclein” is a 140 amino acid protein abundantly expressed in presynaptic terminals of vertebrates. One of its normal functions is to regulate dopamine transporter activities (Wersinger et al., FASEB J. 17: 2151-3, epub Sep. 4, 2003). This protein contains an NAC region that is prone to aggregate, especially under oxidative stress (Ostrerova-Golts et al., J. Neurosci 20: 6048-6054, 2000; Krishnan et al., Biochemistry 42: 829-37, 2003; and Paik et al., FEBS Lett. 537: 63-7, 2003). The aggregated a-synuclein can inhibit the function of 26S proteasome (Conway et al., Ann NY Acad Sci. 920: 42-45, 2000), which is important for the clearance of misfolded proteins and other target molecules. The dysfunction of proteasome will contribute to cell death.

Two mutations, A53T and A30P, in a-synuclein have been identified in families with early-onset familial Parkinson's disease. These mutations can accelerate the aggregation of α-synuclein (Conway et al., Ann NY Acad Sci. 920: 42-45, 2000). It is also interesting to note that, even without mutation, extra copies of the gene encoding α-synuclein can cause Parkinson's disease at an average age of just 34 (Singleton et al., Science 302: 841, 2003).

“α-synuclein”, “β-synuclein”, “γ-synuclein” and “synuclein” are used interchangeably herein.

“Prostaglandin E2”, also referred to as “PGE2” or “PGE₂”, is an important prostaglandin produced in the renal medulla, gastrointestinal mucosa, and other tissues; it causes renal vasodilation and inhibition of renal tubular sodium resorption, inhibition of gastric secretion, and either contraction or relaxation of smooth muscle (depending on the tissue). It is also released by macrophages and modulates several inflammatory responses; it increases vascular permeability, increases pain sensitivity, is pyrogenic, and suppresses lymphocyte transformation, release of mediators from mast cells, and cell-mediated cytotoxicity. PGE2 produced by some tumors causes hypercalcemia by stimulation of bone resorption by osteoclasts.

PGE₂ is a potent autocrine and paracrine factor that is distinct from other eicosanoid products of COX because of the existence of widely expressed multiple receptor subtypes, EP₁, EP₂, EP₃, and EP₄, linked to functionally antagonistic second messenger systems; because of this PGE₂ has versatile and often opposing actions in tissues and cells (reviewed in Breyer et al., Ann Rev Pharmacol Toxicol 41: 661-690, 2001). All of the EP receptors are expressed in rat brain, where there are regional and cell-specific differences in expression and activity. (See Dumont et al., Am J Physiol 275: 1812-1821, 1998; Ek et al., J Comp Neurol 428: 5-20, 2000; Bhattacharya et al., Proc Natl Acad Sci USA 95: 15792-15727, 1998; Nakamura et al., J Comp Neurol 421: 543-569, 2000; Oka et al., J Comp Neurol 428: 20-32, 2000; Sugimoto et al., Neuroscience 62: 919-928, 1994; Zhang et al., Eur J Neurosci 11: 2651-2568, 1997); this is not yet fully characterized in rodents and has not been examined at all in humans. In situ probing, immunohistochemistry, and c-fos expression experiments in rats have shown that EP₁, EP₂, and EP₃ are present in cerebral cortex and hippocampus, the two regions most extensively involved in AD pathogenesis (Ek et al., J Comp Neurol 428: 5-20, 2000; Bhattacharya et al., Proc Natl Acad Sci 95: 15792-15727, 1998; Nakamura et al., J Comp Neurol 421: 543-569, 2000; Oka et al., J Comp Neurol 428: 20-32, 2000; Sugimoto et al., Neuroscience 62: 919-928, 1994) Rodent EP₄ expression is highly restricted to some hypothalamic nuclei (Zhang et al., Eur J Neurosci 11: 2651-2568, 1997). Of these, EP₃ is predominantly expressed on neurons (Dumont et al., Am J Physiol 275: 1812-1821, 1998; Ek et al., J Comp Neurol 428: 5-20, 2000; Nakamura et al., J Comp Neurol 421: 543-569, 2000; Sugimoto et al., Neuroscience 62: 919-928, 1994, whereas EP₁ and EP₂ are expressed on glia (Caggiano et al., J Neurochem 72: 565-575, 1999; Levi et al., Biochimie 80: 899-904, 1998). EP₃ is unlike the other EP receptors in that it has 3 different splice variants, EP₃α, EP₃β, and EP₃γ, EP₃β is unique as it does not desensitize and thus displays persistent signaling when exposed to ligand (Breyer et al., Ann Rev Pharmacol Toxicol 41: 661-690, 2001).

As used interchangeably herein, the terms, a “prostaglandin E2 (PGE2) receptor-mediated activity”, “microglial E-prostanoid (EP) receptor-mediated activity”, “biological activity of the prostaglandin E2 (PGE2) receptor”, “biological activity of the microglial E-prostanoid (EP) receptor”, “functional activity of the “functional activity of the microglial E-prostanoid (EP) receptor””, “functional activity of the microglial E-prostanoid (EP) receptor”, “EP2 receptor-mediated activity”, “biological activity of the PGE2 receptor subtypes”, “functional activity of the EP2 receptor subtype”, or “E-prostanoid (EP) receptor subtype EP2” activity refer to an activity exerted by a prostaglandin (PG) E2 receptor protein, polypeptide or nucleic acid molecule on, e.g., a EP receptor-responsive cell or tissue, or on a EP receptor substrate, ligand, or target molecule, e.g., a protein substrate or target molecule, as determined in vivo, in vitro, or in situ according to standard techniques.

Also as used interchangeably herein, the terms PGE2, EP1, EP2, EP3 and EP4 refer to the prostaglandin E2 (PGE2) receptor or its PGE2 receptor subtypes EP1, EP2, EP3 and EP4.

In one embodiment, a PGE2 receptor activity is a direct activity, such as an association with an EP2 receptor ligand, binding partner, or target molecule. A “ligand”, “binding partner”, or “target molecule” refers to a molecule with which a EP receptor protein binds or interacts in nature, such that a EP receptor-mediated function is achieved. An EP target molecule can be an EP receptor protein or polypeptide, of the present invention or a non-EP receptor. In an exemplary embodiment, a EP2 receptor molecule is an EP2 receptor ligand, e.g., a EP2 receptor ligand.

A EP2 receptor activity can also be an indirect activity, such as an activity mediated by interaction of the EP2 receptor protein with a target molecule such that the target molecule modulates a downstream cellular activity, e.g., a cellular signaling activity modulated indirectly by an interaction of the EP2 receptor protein with a target molecule.

The EP receptor protein sequences and molecules of the invention can modulate the activities of cells in tissues where they are expressed. For example, mRNA are expressed in various cell types including in microglia, astrocytes, and neurons. Accordingly, the PGE2 modulators of the invention can act as therapeutic or diagnostic agents for neurodegenerative disorders.

The PGE2 protein sequences, e.g., proteins, fragments thereof, and derivatives and other variants thereof, can be collectively referred to as “polypeptides or proteins of the invention” or “PGE2 polypeptides or proteins.”Nucleic acid molecules encoding such polypeptides or proteins are collectively referred to as “nucleic acids of the invention” or “PGE2 nucleic acids.”

“Nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA. “Isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

“Hybridizes under stringent conditions” describes conditions for hybridization and washing. Stringent conditions are known to those skilled in the art and can be found in Current Protocols in Molecular Biology, John Wiley & Sons, N.Y., 6.3.1-6.3.6, 1989. Aqueous and nonaqueous methods are described in that reference and either can be used. A preferred, example of stringent hybridization conditions are hybridization in 6× sodium chloride/sodium citrate (SSC) at about 450° C., followed by one or more washes in 0.2× SSC, 0.1 % SDS at 50° C. Another example of stringent hybridization conditions are hybridization in 6× SSC at about 45° C., followed by one or more washes in 0.2× SSC, 0.1% SDS at 55° C. A further example of stringent hybridization conditions are hybridization in 6× SSC at about 45° C., followed by one or more washes in 0.2× SSC, 0.1 % SDS at 60° C. Preferably, stringent hybridization conditions are hybridization in 6× SSC at about 45° C., followed by one or more washes in 0.2× SSC, 0.1% SDS at 65° C. Particularly preferred stringency conditions (and the conditions that should be used if the practitioner is uncertain about what conditions should be applied to determine if a molecule is within a hybridization limitation of the invention) are 0.5 M sodium phosphate, 7% SDS at 65° C., followed by one or more washes at 0.2× SSC, 1% SDS at 65° C. Preferably, an isolated nucleic acid molecule of the invention that hybridizes under stringent conditions to the nucleic acid sequence of a protein of the invention corresponds to a naturally-occurring nucleic acid molecule.

A “naturally-occurring” nucleic acid molecule refers to an RNA or DNA molecule having a nucleotide sequence that occurs in nature (e.g., encodes a natural protein).

“Gene” and “recombinant gene” refer to nucleic acid molecules which include an open reading frame encoding proteins, e.g., PGE2 and the related receptor subtypes, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means a preparation of a protein of the invention having less than about 30%, 20%, 10% and more preferably 5% (by dry weight), of non-proteins of the invention (also referred to herein as a “contaminating protein”), or of chemical precursors or non-chemicals. When the protein, or biologically active portion thereof, is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0. 1, 1.0, and 10 milligrams in dry weight.

A “non-essential” amino acid residue is a residue that can be altered from the wild-type sequence of the protein sequences, e.g., PGE2 and the related receptor subtypes, without abolishing or more preferably, without substantially altering a biological activity, whereas an “essential” amino acid residue results in such a change. For example, amino acid residues that are conserved among the polypeptides of the present invention, are predicted to be particularly not amenable to alteration.

“conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another embodiment, mutations can be introduced randomly along all or part of a coding sequence, such as by saturation mutagenesis, and the resultant mutants can be screened for biological activity to identify mutants that retain activity. Following mutagenesis of the protein sequences, the encoded protein can be expressed recombinantly and the activity of the protein can be determined.

A “biologically active portion” of a protein includes a fragment of a protein which participates in an interaction between a molecule and an effector molecule. Biologically active portions of a protein include peptides comprising amino acid sequences sufficiently identical to or derived from the amino acid sequence of the protein. Typically, biologically active portions comprise a domain or motif with at least one activity of the protein of interest.

A biologically active portion of a protein can be a polypeptide which is, for example, 10, 25, 50, 100, 200, or more, amino acids in length. Biologically active portions of a protein can be used as targets for developing agents which modulate a PGE2 -mediated activity as described herein.

Calculations of homology or sequence identity (the terms are used interchangeably herein) between sequences are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the (Needleman and Wunsch J. Mol. Biol. 48: 444-453, 1970) algorithm which has been incorporated into the GAP program in the GCG software package (available at http:H/www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAβprogram in the GCG software package (available at http:/fwww.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of (E. Meyers and W. Miller CABIOS, 4: 11-17, 1989) which has been incorporated into the ALIGN program (version 2.0), using a PAM 120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of (Altschul, et al. J. Mol. Biol. 215: 403-10, 1990). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in (Altschul et al. Nucleic Acids Res. 25: 3389-3402, 1997). When utilizing BLAST and Gapped BLAST programs, the default parameters of the respective programs (e.g., XBLAST and NBLAST) can be used. See http://www.ncbi.nlm.nih.gov.

Particular polypeptides of the present invention have an amino acid sequence sufficiently identical or substantially identical to the amino acid sequence of the protein sequences. “Sufficiently identical” or “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

“Misexpression or aberrant expression”, as used herein, refers to a non-wild type pattern of gene expression, at the RNA or protein level. It includes: expression at non-wild type levels, i.e., over- or under-expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject” refers to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

2. RNA and DNA Interference Methods

A. Short Interfering RNAs (RNAi)

RNA interference (RNAi) is a mechanism of post-transcriptional gene silencing mediated by double-stranded RNA (dsRNA), which is distinct from antisense and ribozyme-based approaches (see Jain, Pharmacogenomics 5: 239-42, 2004 for a review of RNAi and siRNA). RNA interference is useful in a method for treating a neurodegenerative disease in a mammal by administering to the mammal a nucleic acid molecule (e.g., dsRNA) that hybridizes under stringent conditions to a PGE2 receptor target gene, and attenuates expression of said target gene. dsRNA molecules are believed to direct sequence-specific degradation of mRNA in cells of various types after first undergoing processing by an RNase E-like enzyme called DICER (Bernstein et al., Nature 409: 363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 basepairs long and optionally further comprises one or two single-stranded overhangs or loops. An siRNA can comprise two RNA strands hybridized together, or can alternatively comprise a single RNA strand that includes a self-hybridizing portion. siRNAs can include one or more free strand ends, which can include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, Nat. Biotechnol. 20: 446-448, 2002; See also Yu, J., et al., Proc. Natl. Acad. Sci., 99: 6047-6052, 2002; Sui, et al., Proc. Natl. Acad. Sci USA. 99: 5515-5520, 2002; Paddison, et al., Genes and Dev. 16: 948-958, 2002; Brummelkamp, et al., Science 296: 550-553, 2002; Miyagashi, et al., Nat. Biotech. 20: 497-500, 2002; Paul, et al., Nat. Biotech. 20: 505-508, 2002. As described in these and other references, the siRNA can consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it can also be preferable to select sequences complementary to regions within the 3′ portion of the target transcript. Generally it is preferred to select sequences that contain approximately equimolar ratio of the different nucleotides and to avoid stretches in which a single residue is repeated multiple times.

siRNAs can thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that can be processed in vivo to generate such molecules. Such structures include RNA strands containing two complementary elements that hybridize to one another to form a stem, a loop, and optionally an overhang, preferably a 3′ overhang. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and can be up to approximately 29 nucleotides in length. Loops of 4 nucleotides or greater are less likely subject to steric constraints than are shorter loops and therefore can be preferred. The overhang can include a 5′ phosphate and a 3′ hydroxyl. The overhang can but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript can inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop. (See Grishok, et al., Cell 106: 23-24, 2001; Hutvagner, et al., Science 293: 834-838, 2001; Ketting, et al., Genes Dev., 15: 2654-2659, 2001). Endogenous RNAs of this type have been identified in a number of organisms including mammals, suggesting that this mechanism of post-transcriptional gene silencing can be widespread (Lagos-Quintana, et al., Science 294: 853-858, 2001; Pasquinelli, Trends in Genetics 18: 171-173, 2002, and references in the foregoing two articles). MicroRNAs have been shown to block translation of target transcripts containing target sites in mammalian cells (Zeng, et al., Molecular Cell 9: 1-20, 2002).

siRNAs such as naturally occurring or artificial (i.e., designed by humans) mRNAs that bind within the 3′ UTR (or elsewhere in a target transcript) and inhibit translation can tolerate a larger number of mismatches in the siRNA/template duplex, and particularly can tolerate mismatches within the central region of the duplex. In fact, there is evidence that some mismatches can be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures are possible. For example, the mRNA can include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch can be, for example, 1, 2, 3, or 4 nucleotides in length.

Hairpin structures designed to mimic siRNAs and mRNA precursors are processed intracellularly into molecules capable of reducing or inhibiting expression of target transcripts (McManus, et al., RNA 8: 842-850, 2002). These hairpin structures, which are based on classical siRNAs consisting of two RNA strands forming a 19 bp duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to modulate the expression of a protein in a subject at risk of or suffering from an a neurogenerative disease or disorder and for various of the inventive methods for the identification of compounds for treatment of a neurodegenerative disease or disorder that modulate the activity or level of the molecules described herein. In a preferred embodiment, the therapeutic treatment of a neurodegenerative disease, e.g., AD or PD, with an antibody, antisense vector, or double stranded RNA vector.

The invention therefore provides a method of inhibiting expression of a gene encoding a PGE2 receptor comprising the step of (i) providing a biological system in which expression of a gene encoding PGE2 receptor protein is to be inhibited; and (ii) contacting the system with an siRNA targeted to a transcript encoding the PGE2 receptor protein. According to certain embodiments of the invention the PGE2 receptor protein is encoded by a gene within or linked to an neurodegenerative disease susceptibility locus, or within which a functional mutation causing or contributing to susceptibility or development of an neurodegenerative disease (e.g., AD or PD) can exist. In other embodiments, PGE2 proteins are inhibited. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a transcript encoding any PGE2 receptor proteins (e.g., EP1, EP2, EP3 or EP4). In particular, the invention provides siRNA molecules selectively or specifically targeted to a transcript encoding a polymorphic variant of such a transcript, wherein existence of the polymorphic variant in a subject is indicative of susceptibility to or presence of a degenerative disease (e.g., AD or PD). The terms “selectively” or “specifically targeted to”, in this context, are intended to indicate that the siRNA causes greater reduction in expression of the variant than of other variants (i.e., variants whose existence in a subject is not indicative of susceptibility to or presence of a neurodegenerative disease). The siRNA, or collections of siRNAs, can be provided in the form of kits with additional components as appropriate.

B. Short Hairpin RNAs (shRNA)

RNA interference (RNAi),a mechanism of post-transcriptional gene silencing mediated by double-stranded RNA (dsRNA), is useful in a method for treating a neurodegenerative disease in a mammal by administering to the mammal a nucleic acid molecule (e.g., dsRNA) that hybridizes under stringent conditions to a PGE2 target gene, and attenuates expression of said target gene. See Jain, Pharmacogenomics 5: 239-42, 2004 for a review of RNAi and siRNA. A further method of RNA interference in the present invention is the use of short hairpin RNAs (shRNA). A plasmid containing a DNA sequence encoding for a particular desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, this can be preferable and desired. In cases where longer periods of protein inhibition are necessary, shRNA mediated inhibition is preferable.

C. Full and Partial Length Antisense RNA Transcripts

Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA (Denhardt, Ann N Y Acad. Sci. 660: 70, 1992; Nellen, Trends Biochem. Sci. 18: 419, 1993; Baker et al, Biochem. Biophys. Acta, 1489: 3, 1999; Xu, et al., Gene Therapy 7: 438, 2000; French et al., Curr. Opin. Microbiol. 3: 159, 2000; Terryn et al., Trends Plant Sci. 5: 1360, 2000).

D. Antisense RNA and DNA Oligonucleotides

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but can range from 10 up to approximately 50 nucleotides in length. Binding typically reduces or inhibits the function of the target nucleic acid. For example, antisense oligonucleotides can block transcription when bound to genomic DNA, inhibit translation when bound to mRNA, and/or lead to degradation of the nucleic acid. Reduction in expression of a PGE2 polypeptide can be achieved by the administration of antisense nucleic acids or peptide nucleic acids comprising sequences complementary to those of the mRNA that encodes the polypeptide. Antisense technology and its applications are well known in the art and are described in (Phillips, M. I. (ed.) Antisense Technology, Methods Enzymol., 313 and 314: 2000, and references mentioned therein. See also Crooke, S. “ANTISENSE DRUG TECHNOLOGY: PRINCIPLES, STRATEGIES, AND APPLICATIONS” (1^{st} Edition) Marcel Dekker; and references cited therein.

Antisense oligonucleotides can be synthesized with a base sequence that is complementary to a portion of any RNA transcript in the cell. Antisense oligonucleotides can modulate gene expression through a variety of mechanisms including the modulation of RNA splicing, the modulation of RNA transport and the modulation of the translation of mRNA (Denhardt, Ann N Y Acad. Sci. 660: 70, 1992). Various properties of antisense oligonucleotides including stability, toxicity, tissue distribution, and cellular uptake and binding affinity can be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C) (Wagner, Nat. Medicine 1: 1116, 1995; Varga, et al., Immun. Lett. 69: 217, 1999; Neilsen, Curr. Opin. Biotech. 10: 71, 1999; Woolf, Nucleic Acids Res. 18: 1763, 1990).

The invention provides a method of inhibiting expression of a gene encoding a PGE2 protein comprising the step of (i) providing a biological system in which expression of a gene encoding a PGE2 receptor protein is to be inhibited; and (ii) contacting the system with an antisense molecule that hybridizes to a transcript encoding the PGE2 receptor protein. According to certain embodiments of the invention the PGE2 receptor protein is encoded by a gene within or linked to an a neurodegenerative disease susceptibility locus, or within which a functional mutation causing or contributing to a neurodegenerative disease or development of an neurodegenerative disease (e.g., AD or PD) can exist. In other embodiments, PGE2 receptor proteins are inhibited. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression can be inducible and/or tissue or cell type-specific. The antisense molecule can be an oligonucleotide or a longer nucleic acid molecule. The invention provides such antisense molecules.

E. Ribozymes

Certain nucleic acid molecules referred to as ribozymes or deoxyribozymes have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation (Cotten et al, EMBO J. 8: 3861-3866, 1989; Usman et al., Nucl. Acids Mol. Biol. 10: 243, 1996; Usman, et al., Curr. Opin. Struct. Biol. 1: 527, 1996; Sun, et al., Pharmacol. Rev., 52: 325, 2000. See also e.g., Cotten et al, EMBO J. 8: 3861-3866, 1989).

The invention provides a method of inhibiting expression of a gene encoding a PGE2 receptor protein comprising the step of (i) providing a biological system in which expression of a gene encoding a PGE2 receptor protein is to be inhibited; and (ii) contacting the system with a ribozyme that hybridizes to a transcript encoding the PGE2 receptor protein and directs cleavage of the transcript. According to certain embodiments of the invention the PGE2 receptor protein is encoded by a gene within or linked to a neurodegenerative disease susceptibility locus, or within which a functional mutation causing or contributing to susceptibility or development of an neurodegenerative disease (e.g., AD or PD) can exist. In other embodiments, PGE2 receptor proteins are inhibited. According to certain embodiments of the invention the biological system comprises a cell, and the contacting step comprises expressing the ribozyme in the cell. According to certain embodiments of the invention the biological system comprises a subject, e.g., a mammalian subject such as a mouse or human, and the contacting step comprises administering the ribozyme to the subject or comprises expressing the ribozyme in the subject. The expression can be inducible and/or tissue or cell-type specific according to certain embodiments of the invention. The invention provides ribozymes designed to cleave transcripts encoding PGE2 receptor proteins, or polymorphic variants thereof, as described above.

3. Screening Methodologies

Methods for identifying compounds that inhibit neurodegenerative diseases and disorders can identify compounds that interact with and inhibit PGE2 receptors, e.g., EP1, EP2, EP3 or EP4, and thereby enhancing microglial phagocytosis of pathogenic proteins associated with neurodegenerative diseases (e.g., amyloid beta peptides in the initiation and progression of AD and alpha-synuclein in the initiation and progression of PD). Microglia lacking EP2 receptor display highly desirable dual features of enhanced phagocytosis and clearance of amyloid beta peptides and alpha-synuclein without increased by-stander damage to neurons.

In some embodiments, the test compounds bind to an PGE2 receptor polypeptide or nucleic acid, e.g., mRNA, and cause a decrease in levels of PGE2 receptor polypeptide.

These methods can be used to identify test compounds that inhibit PGE2 receptor function. In some embodiments, the methods include determining whether a compound can bind to a PGE2 receptor and cause the inhibition of the receptor.

In some embodiments, the methods include determining whether a compound that is known to bind to PGE2 also inhibits PGE2 receptor role in neurodegenerative disease, e.g., AD or PD.

In some embodiments, the methods include providing one or more samples that include both PGE2 and one or more test compounds. An “active fragment” is a fragment that retains the ability to bind the other protein, e.g., an active fragment of PGE2 retains the ability to activate key components of PGE2 signaling.

A number of suitable assay methods to detect binding of test compounds to PGE2 receptor protein, e.g., EP2, are known in the art and described herein, and include, but are not limited to, surface plasmon resonance (SPR)/Biacore™, fluorogenic binding assays, fluid phase binding assays, affinity chromatography, size exclusion or gel filtration, ELISA, immunoprecipitation, competitive binding assays, gel shift assays, and mass spectrometry based methods.

In some embodiments, methods described herein include a first screen for compounds that bind to PGE2, e.g., EP2. Compounds that are identified as binding to PGE2 can then be used in a second screen to identify those compounds that inhibit a function of PGE2. Alternatively, the first screen can be omitted and the compounds can simply be screened for their ability to inhibit PGE2 receptors, e.g., EP1, EP2, EP3 or EP4, and thereby enhancing microglial phagocytosis of pathogenic proteins associated with neurodegenerative diseases (e.g., amyloid beta peptides in the initiation and progression of AD and alpha-synuclein in the initiation and progression of PD).

Once a compound that inhibits an action of a PGE2 receptor is identified, the compound can be considered a candidate compound for the treatment of neurodegerative diseases and disorders. The ability of such compounds to treat neurodegenerative diseases and disorders can be evaluated in a population of viable cells or in an animal, e.g., an animal model. A number of methods are known in the art and described herein for measuring PGE2 receptors, e.g., EP2, and thereby enhancing microglial phagocytosis of pathogenic proteins associated with neurodegenerative diseases (e.g., amyloid beta peptides in the initiation and progression of AD and alpha-synuclein in the initiation and progression of PD).

Such compounds are useful, e.g., as candidate therapeutic compounds for the treatment of neurodegerative disease. Thus, included herein are methods for screening for candidate therapeutic compounds for the treatment of neurodegerative disease, as described herein. The methods include administering the compound to a model of the condition, e.g., contacting a cell (in vitro) model with the compound, or administering the compound to an animal model of the condition, e.g., an animal model of a condition associated with neurodegerative disease. The model is then evaluated for an effect of the candidate compound on endpoints or outcome measures that are relevant to neurodegenerative disease including neuron degeneration, neuron death, neuron damage, and glial activation. Examples of such models would be cells in culture, both immortalize and primary cell cultures of glia or neurons, as well as toxic and transgenic models of neurodegenerative diseases. Examples of toxic models of neurodegenerative diseases include systemic exposure to 1-methyl-4-phenyltetrahydropyridine (MTPT) that induces dopaminergic neuron cell death and so models Parkinson's disease. Another toxic model is intracerebroventricular injection of lipopolysaccharide (LPS) to activate innate immunity and paracrine damage to neurons. Transgenic models of neurodegenerations are many and include the expression of mutant genes that are associated with autosomal dominant forms of Alzheimer's disease, Parkinson's disease, amyotrophic lateral sclerosis, Huntington's disease, and prion diseases. Such effects can be determined on a macroscopic or microscopic scale. Methods are such as those described herein. Candidate therapeutic compounds identified by these methods can be further verified, e.g., by administration to human subjects in a clinical trial.

The ability of an agent to inhibit amyloid or synuclein deposition can be evaluated in an animal model system that can be predictive of efficacy in inhibiting amyloid or synuclein deposition in human neurodegenerative diseases, such as a transgenic mouse expressing human APP or other relevant animal models where Aβdeposition is seen. Also contemplated are mouse or other animal models where synuclein disposition is seen. Likewise, the ability of an agent to prevent or reduce cognitive impairment in a model system can be indicative of efficacy in humans.

The test compounds utilized in the assays and methods described herein can be, for example, nucleic acids, small molecules, organic or inorganic compounds, antibodies or antigen-binding fragments thereof, polynucleotides, peptides, or polypeptides. For example, PGE2 protein, e.g., EP2, polypeptides or polynucleotides (e.g., PGE2 polypeptide variants including truncation mutants, deletion mutants, and point mutants; nucleic acids including sense, antisense, aptamers, and small inhibitory RNAs (siRNAs) including short hairpin RNAs (shRNAs) and ribozymes) can be used as test compounds in the methods described herein. Alternatively, compounds or compositions that mimic the PGE2 can be used. A test compound that has been screened by an in vitro method described herein and determined to have a desired activity, can be considered a candidate compound. A candidate compound that has been screened, e.g., in an in vitro or in vivo model, and determined to have a desirable effect on one or more symptoms of a disorder associated with a neurodegenerative disorder, can be considered a candidate therapeutic agent. Candidate therapeutic agents, once screened in a clinical setting, are therapeutic agents, and both types of agents can be optionally optimized (e.g., by derivatization), and formulated with pharmaceutically acceptable excipients or carriers to form pharmaceutical compositions.

Small chemical molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo Solid-Supported Combinatorial and Parallel Synthesis of Small-Molecular-Weight Compound Libraries, Pergamon-Elsevier Science Limited, 1998), and include those such as the “split and pool” or “parallel” synthesis techniques, solid-phase and solution-phase techniques, and encoding techniques (see, for example, Czarnik, Curr. Opin. Chem. Bio., 1: 60, 1997). In addition, a number of small molecule libraries are commercially available.

The test compound can have a structure that is based on an active fragment of PGE2. For example, computer modeling methods known in the can be used to rationally design a molecule that has a structure similar to an active fragment of PGE2.

In some embodiments, the compounds are optimized to improve their therapeutic index, i.e., increase therapeutic efficacy and/or decrease unwanted side effects. Thus, in some embodiments, the methods described herein include optimizing the test or candidate compound. In some embodiments, the methods include formulating a therapeutic composition including a test or candidate compound (e.g., an optimized compound) and a pharmaceutically acceptable carrier. In some embodiments, the compounds are optimized by derivatization using methods known in the art.

In some embodiments, the test compound comprises a polynucleotide that encodes PGE2 protein, e.g., EP2, or an active fragment thereof. In some embodiments, the compound is a polynucleotide that encodes an active fragment of PGE2 protein, e.g., EP2.

In some embodiments, the test compound comprises a polynucleotide that encodes a polypeptide that is at least about 85% identical to the amino acid sequence of PGE2 protein, e.g., EP2. In some embodiments, the polynucleotide encodes a polypeptide that is at least about 90%, 95%, 99%, or 100% identical to the full length sequence of a PGE2 or an active fragment thereof. In some embodiments, the polynucleotide encodes an active peptide fragment thereof that retains the ability to inhibit viral infection by inhibition of virus binding, entry, or replication in cells. In some embodiments, the active fragment is at least about 10, 12, 14, 16, 18, 20, 22, 24, 26, 28, 30, 32, 34, 36, 38, 39, 40, 45, 50, 55, 60, 65, 70, 75, 80 or more amino acids long. The nucleic acid can include one or more noncoding regions of the coding strand of a nucleotide sequence encoding PGE2 protein, e.g., EP2 (e.g., the 5′ and 3′ untranslated regions). A number of methods are known in the art for obtaining suitable nucleic acids, see, e.g., Sambrook et al., Molecular Cloning: A Laboratory Manual (Cold Spring Harbor Laboratory, Cold Spring Harbor, N.Y.; 3rd ed. 2001).

In practicing the methods of the invention, a variety of apparatus and methodologies can be used to in conjunction with the polypeptides and nucleic acids of the invention, e.g., to screen polypeptides for PGE2 activity, to screen compounds as potential modulators (e.g., inhibitors) of a PGE2 activity, for antibodies that bind to a polypeptide of the invention, for nucleic acids that hybridize to a nucleic acid of the invention, to screen for cells expressing a polypeptide of the invention and the like.

In one aspect, the peptides and polypeptides of the invention can be bound to a solid support. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of peptides to a solid support can be direct (i.e., the protein contacts the solid support) or indirect (a particular compound or compounds are bound to the support and the target protein binds to this compound rather than the solid support). Peptides can be immobilized either covalently (e.g., utilizing single reactive thiol groups of cysteine residues (see, e.g., Colliuod, Bioconjugate Chem. 4: 528-536, 1993) or non-covalently but specifically (e.g., via immobilized antibodies (see, e.g., Schuhmann, Adv. Mater. 3: 388-391, 1991; Lu, Anal. Chem. 67: 83-87, 1995); the biotin/strepavidin system (see, e.g., Iwane, Biophys. Biochem. Res. Comm. 230: 76-80, 1997); metal chelating, e.g., Langmuir-Blodgett films (see, e.g., Ng, Langmuir 11: 4048-55, 1995); metal-chelating self-assembled monolayers (see, e.g., Sigal, Anal. Chem. 68: 490-497, 1996) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl) aminobenzoate (SLAB) and the sulfosuccinimidyl derivatives such as sulfosuccinimidyl(4-iodoacetyl) aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce Chemicals, Rockford, Ill.).

Antibodies can be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon, Nature 377: 525-531, 1989).

4. “Arrays or Biochips”

The invention provides methods for identifying/screening for modulators (e.g., inhibitors) of PGE2 receptor activity, e.g., EP2 activity, using arrays. Potential modulators, including small molecules, nucleic acids, polypeptides (including antibodies) can be immobilized to arrays. Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention, e.g., PGE2 activity. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays can be used to simultaneously quantify a plurality of proteins. Small molecule arrays can be used to simultaneously analyze a plurality of PGE2 modulating or binding activities.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts. In practicing the methods of the invention, any known array and/or method of making and using arrays can be incorporated in whole or-in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston, Curr. Biol. 8: R171-R174, 1998; Schummer, Biotechniques 23: 1087-1092, 1997; Kern, Biotechniques 23: 120-124, 1997; Solinas-Toldo, Genes, Chromosomes & Cancer 20: 399-407, 1997; Bowtell, Nature Genetics Supp. 21: 25-32, 1999. See also published U.S. patent applications Nos. 20010018642; 20010019827; 20010016322; 20010014449; 20010014448; 20010012537; 20010008765.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface.

5. Combinational Chemical Libraries

The invention provides methods for identifying/screening for modulators (e.g., inhibitors) of a PGE2 activity, e.g., EP2 activity. In practicing the screening methods of the invention, a test compound is provided. It can be contacted with a polypeptide of the invention in vitro or administered to a cell of the invention or an animal of the invention in vivo. Compounds are also screened using the compositions, cells, non-human animals and methods of the invention for their ability to treat or ameliorate a neurodegenerative disease in an animal. Combinatorial chemical libraries are one means to assist in the generation of new chemical compound leads for, e.g., compounds that inhibit an PGE2 activity of the invention, or a compound that can be used to treat or ameliorate a neurodegenerative disease.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds (see, e.g., Gallop et al. 37: 1233-1250, 1994). Preparation and screening of combinatorial chemical libraries are well known to those of skill in the art, see, e.g., U.S. Pat. No. 6,004,617; 5,985,356. Such combinatorial chemical libraries include, but are not limited to, peptide libraries (see, e.g., U.S. Pat. No. 5,010,175; Furka, Int. J. Pept. Prot. Res. 37: 487-493, 1991, Houghton et al. Nature, 354: 84-88, 1991). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs, Proc. Nat. Acad. Sci. 90: 6909-6913, 1993), vinylogous polypeptides (see, e.g., Hagihara, J. Amer. Chem. Soc. 114: 6568, 1992), non-peptidal peptidomimetics with a Beta-D-Glucose scaffolding (see, e.g., Hirschmann, J. Amer. Chem. Soc. 114: 9217-9218, 1992), analogous organic syntheses of small compound libraries (see, e.g., Chen, J. Amer. Chem. Soc. 116: 2661, 1994), oligocarbamates (see, e.g., Cho, Science 261: 1303, 1993), and/or peptidyl phosphonates (see, e.g., Campbell, J. Org. Chem. 59: 658, 1994). See also Gordon, J. Med. Chem. 37: 1385, 1994), for nucleic acid libraries, peptide nucleic acid libraries, (see, e.g., U.S. Pat. No. 5,539,083; for antibody libraries, see, e.g., Vaughn, Nature Biotechnology 14: 309-314, 1996), for carbohydrate libraries, (see, e.g., Liang et al. Science 274: 1520-1522, 1996), U.S. Pat. No. 5,593,853, for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. Nos. 6,045,755; 5,792,431; 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, PA, Martek Biosciences, Columbia, Md., and the like).

6. Antibodies and Antibody-based Screening Methods

The invention provides isolated or recombinant antibodies that specifically bind to a polypeptide or nucleic acid of the invention, e.g., PGE2 polypeptides, e.g., EP2 polypeptides. These antibodies can be used to isolate, identify or quantify a polypeptide of the invention or related polypeptides. These antibodies can be used to isolate other polypeptides within the scope the invention that act as inhibitors of PGE2 in pathways related to neurodegenerative diseases.

The term “antibody” includes a peptide or polypeptide derived from, modeled after or substantially encoded by an immunoglobulin gene or immunoglobulin genes, or fragments thereof, capable of specifically binding an antigen or epitope, (see, e.g. Fundamental Immunology, Third Edition, W. E. Paul, ed., Raven Press, N.Y., 1993; Wilson, J. Immunol. Methods 175: 267-273, 1994; Yarmush, J. Biochem. Biophys. Methods 25: 85-97, 1992). The term antibody includes antigen-binding portions, i.e., “antigen binding sites,” (e.g., fragments, subsequences, complementarity determining regions (CDRs) that retain capacity to bind antigen, including (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). Single chain antibodies are also included by reference in the term “antibody.”

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, (see, e.g., Coligan, Current Protocols in Immunology, 1991; Stites Basic and Clinical Immunology 7: 1986; Kohler, Nature 256: 495, 1975; Harlow, Antibodies: A Laboratory Manual, 1988). Antibodies also can be generated in vitro, e.g., using recombinant antibody binding site expressing phage display libraries, in addition to the traditional in vivo methods using animals. See, e.g., Hoogenboom, Trends Biotechnol. 15: 62-70, 1997; Katz, Annu. Rev. Biophys. Biomol. Struct. 26: 27-45, 1997.

Polypeptides or peptides can be used to generate antibodies which bind specifically to the polypeptides of the invention. The resulting antibodies can be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody can be determined using any of a variety of procedures familiar to those skilled in the art. For example, binding can be determined by labeling the antibody with a detectable label such as a fluorescent agent, an enzymatic label, or a radioisotope. Alternatively, binding of the antibody to the sample can be detected using a secondary antibody having such a detectable label thereon. Particular assays include ELISA assays, sandwich assays, radioimmunoassay, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which can bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole, Monoclonal Antibodies and Cancer Therapy, 77-96, 1985).

Techniques described for the production of single chain antibodies (see, e.g., U.S. Pat. No. 4,946,778) can be adapted to produce single chain antibodies to the polypeptides of the invention. Alternatively, transgenic mice can be used to express humanized antibodies to these polypeptides or fragments thereof.

Antibodies generated against the polypeptides of the invention can be used in screening for similar polypeptides from other organisms and samples. In such techniques, polypeptides from the organism are contacted with the antibody and those polypeptides which specifically bind the antibody are detected. Any of the procedures described above can be used to detect antibody binding.

7. PGE2 Receptor Inhibitors

The present invention provides a method for preventing or treating neurodegenerative diseases or disorders in a mammalian subject comprises administering to the mammalian subject a compound capable of inhibiting a PGE2 receptor, wherein the compound is administered in an amount effective to reduce or eliminate the neurodegenerative disease or to prevent its occurrence or recurrence. The present invention further provides a method for identifying a compound capable of inhibiting neurodegerative disease comprising contacting a test compound with a cell-based assay system comprising a cell expressing PGE2 and capable of signaling responsiveness to PGE2, and detecting an effect of the test compound on PGE2 signaling in the assay system as an increase or a decrease in susceptibility of the cell line to neurodegenerative disease, effectiveness of the test compound in the assay being indicative of the inhibition of PGE2.

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

The term “patient” or “subject”, as used herein, refers to any individual having symptoms of, or at risk for, any neurodegenerative disease (e.g., for example, Alzheimer's disease, Parkinson's disease, cystic fibrosis or bovine spongiform encephalopathy). For example, it is known that patients can be at risk for Alzheimer's or Parkinson's disease because of family history (i.e., indicative of genetic predisposition). A patient or subject can be human or non-human and can include, for example, animal strains or species used as “model systems” for research purposes.

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., a neurodegenerative disease). “Treating” further refers to any indicia of success in the treatment or amelioration or prevention of the disease, condition, or disorder (e.g., a neurodegenerative disease), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a neurodegenerative disease. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a neurodegenerative disease but does not yet experience or exhibit symptoms, inhibiting the symptoms of a neurodegenerative disease (slowing or arresting its development), providing relief from the symptoms or side-effects of neurodegenerative disease (including palliative treatment), and relieving the symptoms of neurodegenerative disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

The ability of a molecule to bind to PGE2 can be determined, for example, by the ability of the putative ligand to bind to PGE2 immunoadhesin coated on an assay plate. Specificity of binding can be determined by comparing binding to non- PGE2 immunoadhesin.

In one embodiment, antibody binding to PGE2 can be assayed by either immobilizing the ligand or the receptor. For example, the assay can include immobilizing PGE2 fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

“Modulator” includes inhibitors and activators. Inhibitors are agents that, e.g., bind to, partially or totally block stimulation, decrease, prevent, delay activation, inactivate, desensitize, or down regulate the activity of PGE2, e.g., antagonists. Activators are agents that, e.g., bind to, stimulate, increase, open, activate, facilitate, enhance activation, sensitize or up regulate the activity of PGE2, e.g., agonists. Modulators include agents that, e.g., alter the interaction of PGE2 with proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring PGE2 ligands, e.g., with altered activity, as well as naturally occurring and synthetic ligands, antagonists, agonists, small chemical molecules and the like. Such assays for inhibitors and activators include, e.g., applying putative modulator compounds to a cell expressing a PGE2 and then determining the functional effects on PGE2 activity, as described herein. Samples or assays comprising PGE2 that are treated with a potential activator, inhibitor, or modulator are compared to control samples without the inhibitor, activator, or modulator to examine the extent of inhibition. Control samples (untreated with inhibitors) can be assigned a relative PGE2 receptor activity value of 100%. Inhibition of PGE2 receptor is achieved when the PGE2 receptor activity value relative to the control is about 80%, optionally 50% or 25-0%. Activation of PGE2 receptor is achieved when the PGE2 receptor activity value relative to the control is 110%, optionally 150%, optionally 200-500%, or 1000-3000% higher.

“Inhibitors,” “activators,” and “modulators” of PGE2 protein, e.g., EP2, activity are used to refer to inhibitory, activating, or modulating molecules, respectively, identified using in vitro and in vivo assays for PGE2 activity, e.g., ligands, agonists, antagonists, and their homologs and mimetics. “Modulator” includes inhibitors and activators. Inhibitors are agents that, e.g., bind to, partially or totally block stimulation, decrease, prevent, delay activation, inactivate, desensitize, or down regulate the activity of PGE2, e.g., antagonists. Activators are agents that, e.g., bind to, stimulate, increase, open, activate, facilitate, enhance activation, sensitize or up regulate the activity of PGE2, e.g., agonists. Modulators include agents that, e.g., alter the interaction of PGE2 with proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring PGE2 protein, e.g., EP2, with altered activity, as well as naturally occurring and synthetic ligands, antagonists, agonists, small chemical molecules and the like. Such assays for inhibitors and activators include, e.g., applying putative modulator compounds to a cell expressing PGE2 protein, e.g., EP2, and then determining the functional effects on viral infection in the cell, as described herein. Samples or assays comprising PGE2 protein, e.g., EP2 that are treated with a potential activator, inhibitor, or modulator are compared to control samples without the inhibitor, activator, or modulator to examine the extent of inhibition. Control samples (untreated with inhibitors) can be assigned a relative PGE2 activity value of 100%. Inhibition of viral infection is achieved when the PGE2 activity value relative to the control is about 80%, optionally 50% or 25-0%.

“Antagonist” is used in the broadest sense, and includes any molecule that partially or fully blocks, inhibits, or neutralizes a biological activity of PGE2 protein, e.g., EP2, activity. In a similar manner, the term “agonist” is used in the broadest sense and includes any molecule that mimics or enhances a biological activity of PGE2 protein, e.g., EP2, activity. Suitable agonist or antagonist molecules specifically include agonist or antagonist antibodies or antibody fragments, fragments or amino acid sequence variants of native PGE2 polypeptides, peptides, antisense oligonucleotides, small organic molecules, and the like. Methods for identifying agonists or antagonists of PGE2 polypeptides can comprise contacting an PGE2 polypeptide with a candidate agonist or antagonist molecule and measuring a detectable change in one or more biological activities normally associated with the PGE2.

“Test compound” refers to a nucleic acid, DNA, RNA, protein, polypeptide, or small chemical entity that is determined to effect an increase or decrease in a gene expression as a result of signaling through PGE2 protein, e.g., EP2. The test compound can be an antisense RNA, ribozyme, polypeptide, or small molecular chemical entity. The term “test compound” can be any small chemical compound, or a biological entity, such as a protein, sugar, nucleic acid or lipid. Typically, test compounds will be small chemical molecules and polypeptides. A “test compound specific for signaling through PGE2” is determined to be a modulator of PGE2 activity.

“Cell-based assays” include PGE2 binding assays, for example, radioligand or fluorescent ligand binding assays for PGE2 activity, e.g., EP2, in cells, plasma membranes, detergent-solubilized plasma membrane proteins, immobilized collagen (Alberdi, J Biol Chem. 274: 31605-12, 1999; Meyer et al., 2002); PGE2 -affinity column chromatography (Alberdi, J Biol Chem. 274: 31605-12, 1999; Aymerich et al., Invest Ophthalmol Vis Sci. 42: 3287-93, 2001); PGE2 ligand blot using a radio- or fluorosceinated-ligand (Aymerich et al., Invest Ophthalmol Vis Sci. 42: 3287-93, 2001; Meyer et al., 2002); Size-exclusion ultrafiltration (Alberdi et al., Biochem., 1998; Meyer et al., 2002); or ELISA.

In one embodiment, PGE2 protein, e.g., EP2, can be assayed by either immobilizing the ligand or the receptor. For example, the assay can include immobilizing PGE2 fused to a His tag onto Ni-activated NTA resin beads. Inhibitors of PGE2 can be added in an appropriate buffer and the beads incubated for a period of time at a given temperature. After washes to remove unbound material, the bound protein can be released with, for example, SDS, buffers with a high pH, and the like and analyzed.

“Contacting” refers to mixing a test compound in a soluble form into an assay system, for example, a cell-based assay system, such that an effect upon receptor-mediated signaling can be measured.

“Signaling in cells” refers to the interaction of a ligand with a receptor, such as a PGE2 receptor, e.g., EP2 receptor subtype, to produce a response, for example, such as enhanced phagocytosis and clearance of amyloid beta peptides and alpha-synuclein without increased by-stander damage to neurons. “Signaling responsiveness” or “effective to activate signaling” or “stimulating a cell-based assay system” refers to the ability of inhibitors of PGE2 activity to stimulate an innate immune response, and to prevent or alleviate a neurodegenerative disease.

“Detecting an effect” refers to an effect measured in a cell-based assay system. For example, the effect detected can be PGE2 protein activity, e.g., EP2, in an assay system, for example, an in vitro assay.

“Assay being indicative of modulation” refers to results of a cell-based assay system indicating that cell activation by PGE2 protein, e.g., EP2, induces a protective response in cells against a neurodegenerative disease.

“Biological activity” and “biologically active” with regard to an inhibitor of PGE2 protein, e.g., EP2, of the present invention refer to the ability of the inhibitor molecule to specifically bind to and signal through a native or recombinant PGE2, or to block the ability of a native or recombinant PGE2 to participate in signal transduction. Thus, the (native and variant) ligands of PGE2 of the present invention include agonists and antagonists of a native or recombinant PGE2. Preferred biological activities of the ligands of PGE2 protein, e.g., EP2, of the present invention include the ability to suppress an immune response, augment phagocytosis, or treat a neurodegenerative disease or disorder. Accordingly, the administration of the compounds or agents of the present invention can prevent or delay, to alleviate, or to arrest or inhibit development of the symptoms or conditions associated with a neurodegenerative disease, or other disorders.

“Concomitant administration” of a known drug with a compound of the present invention means administration of the drug and the compound at such time that both the known drug and the compound will have a therapeutic effect or diagnostic effect. Such concomitant administration can involve concurrent (i.e., at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

In general, the phrase “well tolerated” refers to the absence of adverse changes in health status that occur as a result of the treatment and would affect treatment decisions.

“Epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An intact “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) through cellular receptors such as Fc receptors (e.g., FcγRI, FcγRIIa, FcγRIIb, FcγRIII, and FcRη) and the first component (Clq) of the classical complement system. The term antibody includes antigen-binding portions of an intact antibody that retain capacity to bind the antigen. Examples of antigen binding portions include (i) a Fab fragment, a monovalent fragment consisting of the V_{L}, V_{H}, CL and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); See, e.g., Bird et al., Science 242: 423-426, 1988; and Huston et al., Proc. Natl. Acad. Sci. U.S.A. 85: 5879-5883, 1988). Such single chain antibodies are included by reference to the term “antibody” Fragments can be prepared by recombinant techniques or enzymatic or chemical cleavage of intact antibodies.

“Human sequence antibody” includes antibodies having variable and constant regions (if present) derived from human immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). However, the term “human sequence antibody”, as used herein, is not intended to include antibodies in which entire CDR sequences sufficient to confer antigen specificity and derived from the germline of another mammalian species, such as a mouse, have been grafted onto human framework sequences (i.e., humanized antibodies).

“Monoclonal antibody” or “monoclonal antibody composition” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Diclonal antibody” refers to a preparation of at least two antibodies to an antigen. Typically, the different antibodies bind different epitopes.

“Oligoclonal antibody” refers to a preparation of 3 to 100 different antibodies to an antigen. Typically, the antibodies in such a preparation bind to a range of different epitopes.

“Polyclonal antibody” refers to a preparation of more than 1 (two or more) different antibodies to an antigen. Such a preparation includes antibodies binding to a range of different epitopes.

“Recombinant human antibody” includes all human sequence antibodies of the invention that are prepared, expressed, created or isolated by recombinant means, such as antibodies isolated from an animal (e.g., a mouse) that is transgenic for human immunoglobulin genes (described further below); antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, can not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

A “heterohybrid antibody” refers to an antibody having a light and heavy chains of different organismal origins. For example, an antibody having a human heavy chain associated with a murine light chain is a heterohybrid antibody.

“Substantially pure” or “isolated” means an object species (e.g., an antibody of the invention) has been identified and separated and/or recovered from a component of its natural environment such that the object species is the predominant species present (i.e., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., antibodies of the invention) can also be purified to essential homogeneity (contaminant species cannot be detected in the composition by conventional detection methods) wherein the composition consists essentially of derivatives of a single macromolecular species. For example, an isolated antibody to PGE2 can be substantially free of other antibodies that lack binding to human PGE2 and bind to a different antigen. Further, an isolated antibody that specifically binds to an epitope, isoform or variant of human PGE2 can, however, have cross-reactivity to other related antigens, e.g., from other species (e.g., PGE2 species homologs). Moreover, an isolated antibody of the invention be substantially free of other cellular material (e.g., non-immunoglobulin associated proteins) and/or chemicals.

“Specific binding” refers to preferential binding of an antibody to a specified antigen relative to other non-specified antigens. The phrase “specifically (or selectively) binds” to an antibody refers to a binding reaction that is determinative of the presence of the protein in a heterogeneous population of proteins and other biologics. Typically, the antibody binds with an association constant (Kₐ) of at least about 1×10⁶ M⁻¹ or 10⁷ M⁻¹, or about 10⁸ M⁻¹ to 10⁹ M⁻¹, or about 10¹⁰ M⁻¹ to 10¹¹ M⁻¹ or higher, and binds to the specified antigen with an affinity that is at least two-fold greater than its affinity for binding to a non-specific antigen (e.g., BSA, casein) other than the specified antigen or a closely-related antigen. The phrases “an antibody recognizing an antigen” and “an antibody specific for an antigen” are used interchangeably herein with the term “an antibody which binds specifically to an antigen”. A predetermined antigen is an antigen that is chosen prior to the selection of an antibody that binds to that antigen.

“Specifically bind(s)” or “bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore™ and Western blot are used to identify peptides that specifically react with the antigen. Typically a specific or selective reaction will be at least twice background signal or noise and more typically more than 10 times background.

“High affinity” for an antibody refers to an equilibrium association constant (Kₐ) of at least about 10⁷M⁻¹, at least about 10⁸M⁻¹, at least about 10⁹M⁻¹, at least about 10¹⁰M⁻¹, at least about 10¹¹M⁻¹, or at least about 10¹²M⁻¹ or greater, e.g., up to 10¹³M⁻¹ or 10¹⁴M⁻¹ or greater. However, “high affinity” binding can vary for other antibody isotypes.

The term “K^{a}”, as used herein, is intended to refer to the equilibrium association constant of a particular antibody-antigen interaction. This constant has units of 1/M.

The term “K_{d}”, as used herein, is intended to refer to the equilibrium dissociation constant of a particular antibody-antigen interaction. This constant has units of M.

The term “kₐ”, as used herein, is intended to refer to the kinetic association constant of a particular antibody-antigen interaction. This constant has units of 1/Ms.

The term “k_{d}”, as used herein, is intended to refer to the kinetic dissociation constant of a particular antibody-antigen interaction. This constant has units of 1/s.

“Particular antibody-antigen interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

“Isotype switching” refers to the phenomenon by which the class, or isotype, of an antibody changes from one Ig class to one of the other Ig classes.

“Nonswitched isotype” refers to the isotypic class of heavy chain that is produced when no isotype switching has taken place; the C_{H} gene encoding the nonswitched isotype is typically the first C_{H} gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching can occur by, for example, homologous recombination between human σ_{μ} and human Σ_{μ} (δ-associated deletion). Alternative non-classical switching mechanisms, such as intertransgene and/or interchromosomal recombination, among others, can occur and effectuate isotype switching.

“Switch sequence” refers to those DNA sequences responsible for switch recombination. A “switch donor” sequence, typically a μ switch region, are 5′ (i.e., upstream) of the construct region to be deleted during the switch recombination. The “switch acceptor” region are between the construct region to be deleted and the replacement constant region (e.g., γ, ε, and alike). As there is no specific site where recombination always occurs, the final gene sequence is not typically predictable from the construct.

“Glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the non-human transgenic animal, when one of ordinary skill in the art would recognize the glycosylation pattern of the heterologous antibody as being more similar to said pattern of glycosylation in the species of the non-human transgenic animal than to the species from which the C_{H} genes of the transgene were derived.

“Naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Immunoglobulin locus” refers to a genetic element or set of linked genetic elements that comprise information that can be used by a B cell or B cell precursor to express an immunoglobulin peptide. This peptide can be a heavy chain peptide, a light chain peptide, or the fusion of a heavy and a light chain peptide. In the case of an unrearranged locus, the genetic elements are assembled by a B cell precursor to form the gene encoding an immunoglobulin peptide. In the case of a rearranged locus, a gene encoding an immunoglobulin peptide is contained within the locus.

“Rearranged” refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus has at least one recombined heptamer/nonamer homology element.

“Unrearranged” or “germline configuration” in reference to a V segment refers to the configuration wherein the V segment is not recombined so as to be immediately adjacent to a D or J segment.

“Nucleic acid” or “nucleic acid molecule” refer to a deoxyribonucleotide or ribonucleotide polymer in either single- or double-stranded form, and unless otherwise limited, can encompass known analogs of natural nucleotides that can function in a similar manner as naturally occurring nucleotides.

“Isolated nucleic acid” in reference to nucleic acids encoding antibodies or antibody portions (e.g., V_{H}, V_{L}, CDR3) that bind to the antigen, is intended to refer to a nucleic acid in which the nucleotide sequences encoding the antibody or antibody portion are free of other nucleotide sequences encoding antibodies or antibody portions that bind antigens other than, for example, PGE2, which other sequences can naturally flank the nucleic acid in human genomic DNA.

“Substantially identical,” in the context of two nucleic acids or polypeptides refers to two or more sequences or subsequences that have at least about 80%, about 90%, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, or over a region at least about 150 residues, or over the full length of the two sequences to be compared. As described below, any two antibody sequences can only be aligned in one way, by using the numbering scheme in Kabat. Therefore, for antibodies, percent identity has a unique and well-defined meaning.

Amino acids from the variable regions of the mature heavy and light chains of immunoglobulins are designated Hx and Lx respectively, where x is a number designating the position of an amino acid according to the scheme of (Kabat, Sequences of Proteins of Immunological Interest, 1987 and 1991). Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention. An alternative structural definition has been proposed by Chothia, et al., J. Mol. Biol. 196: 901-917, 1987; Chothia, et al., Nature 342: 878-883, 1989; and Chothia, et al., J. Mol. Biol. 186: 651-663, 1989, which are herein incorporated by reference for all purposes.

The nucleic acids of the invention be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook et al., Molecular Cloning, A Laboratory Manual, 2^{nd} ed., 1989; Tijssen, 1993; and Ausubel, 1994, incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, (see, e.g., Sambrook, et al., 1989). Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The nucleic acid compositions of the present invention, while often in a native sequence (except for modified restriction sites and the like), from either cDNA, genomic or mixtures can be mutated, thereof in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Recombinant host cell” or “host cell” refers to a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny can not, in fact, be identical to the parent cell, but are still included within the scope of the term “host cell” as used herein.

A “label” is a composition detectable by spectroscopic, photochemical, biochemical, immunochemical, or chemical means. For example, useful labels include ³²p, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines measured by standard methods (Windhagen A; et al., Immunity 2: 373-80, 1995), (3) antigen presenting cells can be incubated with whole protein antigen and the presentation of that antigen on MHC detected by either T lymphocyte activation assays or biophysical methods (Harding et al.., Proc. Natl. Acad. Sci. U.S.A., 86: 4230-4, 1989), (4) mast cells can be incubated with reagents that cross-link their Fc-epsilon receptors and histamine release measured by enzyme immunoassay (Siraganian, et al., TIPS 4: 432-437, 1983).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood 72: 1310-5, 1988); (3) the proliferation of peripheral blood mononuclear cells in response to mitogens or mixed lymphocyte reaction can be measured using ³H-thyrmidine; (4) the phagocytic capacity of granulocytes, macrophages, and other phagocytes in PBMCs can be measured by placing PMBCs in wells together with labeled particles (Peters et al., 1988); and (5) the radioimmunoassay of immune system cells can be measured by labeling PBMCs with antibodies to CD molecules such as CD4 and CD8 and measuring the fraction of the PBMCs expressing these markers.

“Signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response, e.g., an immune response described above.

A signal transduction pathway refers to the biochemical relationship between a variety of signal transduction molecules that play a role in the transmission of a signal from one portion of a cell to another portion of a cell. As used herein, the phrase “cell surface receptor” includes molecules and complexes of molecules capable of receiving a signal and the transmission of such a signal across the plasma membrane of a cell. An example of a “cell surface receptor” is the PGE2 receptor.

A signal transduction pathway in a cell can be initiated by interaction of a cell with a stimulator that is inside or outside of the cell. If an exterior (i.e., outside of the cell) stimulator (e.g., an MHC-antigen complex on an antigen presenting cell) interacts with a cell surface receptor (e.g., a T cell receptor), a signal transduction pathway can transmit a signal across the cell's membrane, through the cytoplasm of the cell, and in some instances into the nucleus. If an interior (e.g., inside the cell) stimulator interacts with an intracellular signal transduction molecule, a signal transduction pathway can result in transmission of a signal through the cell's cytoplasm, and in some instances into the cell's nucleus.

Signal transduction can occur through, e.g., the phosphorylation of a molecule; non-covalent allosteric interactions; complexing of molecules; the conformational change of a molecule; calcium release; inositol phosphate production; proteolytic cleavage; cyclic nucleotide production and diacylglyceride production. Typically, signal transduction occurs through phosphorylating a signal transduction molecule.

In accordance with the present invention, there can be employed conventional molecular biology, microbiology, immunology, and recombinant DNA techniques within the skill of the art. Such techniques are explained fully in the literature. Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, Fitsch & Maniatis, 1989, MOLECULAR CLONING: A LABORATORY MANUAL, 2^{nd}, Cold Spring Harbor Laboratory Press, Cold Spring Harbor, N.Y. (referred to herein as “Sambrook et al., 1989”); 1989; Kriegler, GENE TRANSFER AND EXPRESSION: A LABORATORY MANUAL, 1990; DNA Cloning: A Practical Approach, Volumes I and II (D. N. Glover ed. 1985); Oligonucleotide Synthesis (M. J. Gait ed. 1984); NUCLEIC ACID HYBRIDIZATION (Hames, B.D. & S. J. Higgins, eds. 1984); Animal Cell Culture (R. I. Freshney, ed. 1986); IMMOBILIZED CELLS AND ENZYMES (IRL Press, 1986); B. E. Perbal, 1984, A PRACTICAL GUIDE TO MOLECULAR CLONING; F. M. Ausubel et al. (eds.), CURRENT PROTOCOLS IN MOLECULAR BIOLOGY, 1997, John Wiley & Sons, Inc., N. C. Dracopoli et al. (eds.), CURRENT PROTOCOLS IN HUMAN GENETICS, 1997, John Wiley & Sons, Inc., A. D. Baxevanis et al. (eds.), CURRENT PROTOCOLS IN BIOINFORMATICS, 1992, John Wiley & Sons, Inc.; LABORATORY TECHNIQUES IN BIOCHEMISTRY AND MOLECULAR BIOLOGY: HYBRIDIZATION WITH NUCLEIC ACID PROBES, PART I. THEORY AND NUCLEIC ACID PREPARATION, Tijssen, ed. Elsevier, N.Y., 1993 (these references are herein incorporated by reference in their entirety for all purposes).

PGE2, e.g., EP2, nucleic acids, polymorphic variants, orthologs, and alleles that are substantially identical to sequences provided herein can be isolated using nucleic acid probes and oligonucleotides of PGE2, e.g., EP2, under stringent hybridization conditions, by screening libraries. Alternatively, expression libraries can be used to PGE2 protein, or protein encoding PGE2 polymorphic variants, orthologs, and alleles by detecting expressed homologs immunologically with antisera or purified antibodies made against human PGE2, or portions thereof.

8. Peptides and Polypeptides

The invention provides methods for inhibiting the activity of PGE2 polypeptide, e.g., EP2 polypeptides, e.g., a polypeptide of the invention. The invention also provides methods for screening for compositions that inhibit the activity of, or bind to (e.g., bind to the active site), of PGE2 polypeptides, e.g., a polypeptide of the invention.

In one aspect, the invention provides PGE2 polypeptides (and the nucleic acids encoding them) where one, some or all of the PGE2 polypeptides replacement with substituted amino acids. In one aspect, the invention provides methods to disrupt the interaction of PGE2 polypeptides with other proteins, in pathways related to neurodegenerative diseases or conditions.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a PGE2 activity and for agents that can treat or ameliorate an neurodegenerative disease.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, A. K., THERAPEUTIC PEPTIDES AND PROTEINS, FORMULATION, PROCESSING AND DELIVERY SYSTEMS, Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has a PGE2 activity. A mimetic composition can also be within the scope of the invention if it can inhibit an activity of a PGE2 polypeptide, e.g., EP2 polypeptides of the invention, e.g., be a dominant negative mutant or, bind to an antibody of the invention.

In one aspect, the polypeptide or peptidomimetic composition can be a dominant-negative mutant within the scope of the invention if it can inhibit an activity of a PGE2 polypeptide, e.g., EP2 polypeptides of the invention, e.g., be a dominant-negative mutant or bind to an antibody of the invention. The dominant negative mutant can be a peptide or peptide mimetic that can inhibit an activity of a PGE2, or a nucleic acid composition, in the form of a DNA vector or gene therapy vector, that expresses a dominant-negative polypeptide that can inhibit an activity of a PGE2. The dominant negative mutant can bind to a ligand of PGE2 or a target, affecting ligand target interaction. The dominant negative molecule can act, for example, by blocking protein protein interactions.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., ——C(═O)—CH₂— for —C(═O)—NH——), aminomethylene (CH₂—NH), ethylene, olefin (CH═CH), ether (CH₂—O), thioether (CH₂—S), tetrazole (CN₄——), thiazole, retroamide, thioamide, or ester (see, e.g., Spatola, in Chemistry and Biochemistry of Amino Acids, Peptides and Proteins, Vol. 7, pp 267-357, “Peptide Backbone Modifications,” Marcell Dekker, NY, 1983).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or guanidino-acetic acid, or (guanidino)alkyl-acetic acid, where alkyl is defined above. Nitrile derivative (e.g., containing the CN-moiety in place of COOH) can be substituted for aspargine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of □adioim include, e.g., pipecolic acid, thiazolidine carboxylic acid, 3- or 4-hydroxy radioimmuno, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of radioimmuno and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form.

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a PGE2 polypeptide of the invention, resulting in modification of the structure of the polypeptide, without significantly altering its biological activity. For example, amino- or carboxyl-terminal, or internal, amino acids which are not required for a PGE2 activity can be removed.

The skilled artisan will recognize that individual synthetic residues and polypeptides incorporating these mimetics can be synthesized using a variety of procedures and methodologies, which are WELL DESCRIBED IN THE SCIENTIFIC AND PATENT LITERATURE, E.G., ORGANIC Syntheses Collective Volumes, Gilman, et al. (eds.) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; (see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996). Modified peptides of the invention can be further produced by chemical modification methods, (see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994).

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Inc., Seattle, Wash.). The inclusion of a cleavable linker sequences such as Factor Xa or enterokinase (Invitrogen, San Diego, Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, (see e.g., Kroll, DNA Cell. Biol., 12: 441-53, 1993).

“Polypeptide” and “protein” as used herein, refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

“Isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

9. Prodrugs

The present invention is also related to prodrugs of the agents obtained by the methods disclosed herein. Prodrugs are agents which are converted in vivo to active forms (see, e.g., R. B. Silverman, 1992, THE ORGANIC CHEMISTRY OF DRUG DESIGN AND DRUG ACTION, Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties can be metabolized in vivo by esterases or by other mechanisms to carboxylic acids.

Examples of prodrugs and their uses are well known in the art (see, e.g., Berge et al., “Pharmaceutical Salts”, J. Pharm. Sci. 66: 1-19, 1977). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

10. Therapeutic Applications

The small chemical molecule, siRNA molecule, dominant-negative mutants, or antibody inhibitors of PGE2 identified by the methods of the present invention can be used in a variety of methods of treatment. Thus, the present invention provides compositions and methods for treating a neurodegerative disease or disorder, e.g., Alzheimer's disease (“AD”) or Parkinson's disease (“PD”).

Exemplary neurodegenerative diseases, include but are not limited to age-related cognitive decline, early Alzheimer's disease as seen in Mild Cognitive Impairment (“MCI”), vascular dementia, or Alzheimer's disease (“AD”), which can be sporadic (non-hereditary) Alzheimer's disease or familial (hereditary) Alzheimer's disease. The neurodegenerative diseases can also be cerebral amyloid angiopathy (“CAA”) or hereditary cerebral hemorrhage, senile dementia, Down's syndrome, inclusion body myositis (“IBM”), or age-related macular degeneration (“ARMD”). Other neurodegenerative diseases or disorders include but are not limited to those involving Lewy bodies, such as dementia with Lewy bodies, multiple system atrophy, and Hallervorden-Spatz disease.

Preferably, treatment using PGE2 inhibitors, e.g., a small chemical molecule inhibitor, a polypeptide inhibitor, or a peptidomimetic inhibitor of replication of the present invention could either be by administering an effective amount of the small chemical molecule inhibitor, the polypeptide inhibitor, or the peptidomimetic inhibitor to the patient, or by removing cells from the patient, supplying the cells with a polynucleotide of the present invention, and returning the engineered cells to the patient (ex vivo therapy).

11. Blood-Brain Barrier

Nitric oxide is a vasodilator of the peripheral vasculature in normal tissue of the body. Increasing generation of nitric oxide by nitric oxide synthase causes vasodilation without loss of blood pressure. The blood-pressure-independent increase in blood flow through brain tissue increases cerebral bioavailability of blood-born compositions. This increase in nitric oxide can be stimulated by administering L-arginine. As nitric oxide is increased, cerebral blood flow is consequently increased, and drugs in the blood stream are carried along with the increased flow into brain tissue. Therefore, L-arginine may be used in the pharmaceutical compositions of the invention to enhance delivery of agents to brain tissue after introducing a pharmaceutical composition into the blood stream of the subject substantially contemporaneously with a blood flow enhancing amount of L-arginine. WO 00/56328

Agents of the invention that exert their physiological effect in vivo in the brain can be more useful if they gain access to target cells in the brain. Non-limiting examples of brain cells are neurons, glial cells (astrocytes, oligodendrocytes, microglia), cerebrovascular cells (muscle cells, endothelial cells), and cells that comprise the meninges. The blood brain barrier (“BBB”) typically restricts access to brain cells by acting as a physical and functional blockade that separates the brain parenchyma from the systemic circulation (see, e.g., Pardridge et al., J. Neurovirol. 5: 556-69, 1999; Rubin et al., Rev. Neurosci. 22: 11-28, 1999). Circulating molecules are normally able to gain access to brain cells via one of two processes: lipid-mediated transport through the BBB by free diffusion, or active (or catalyzed) transport.

The agents of the invention can be formulated to improve distribution in vivo, for example as powdered or liquid tablet or solution for oral administration or as a nasal spray, nose drops, a gel or ointment, through a tube or catheter, by syringe, by packtail, by pledget, or by submucosal infusion. For example, the blood-brain barrier (BBB) excludes many highly hydrophilic agents. To ensure that the more hydrophilic therapeutic agents of the invention cross the BBB, they can be formulated, for example, in liposomes. For methods of manufacturing liposomes, (see, e.g., U.S. Pat. Nos. 4,522,811; 5,374,548; and 5,399,331). The liposomes can comprise one or more moieties which are selectively transported into specific cells or organs (“targeting moieties” or “targeting groups” or “transporting vectors”), thus providing targeted drug delivery (see, e.g., Ranade, J. Clin. Pharmacol. 29: 685, 1989). Likewise, the agents can be linked to targeting groups that facilitate penetration of the blood brain barrier. In one embodiment, the method of the present invention employs a naturally occurring polyamine linked to an agent that is a small molecule and is useful for inhibiting AO deposition.

To facilitate transport of agents of the invention across the BBB, they can be coupled to a BBB transport vector (for review of BBB transport vectors and mechanisms, see, Bickel et al., Adv. Drug Delivery Reviews 46: 247-79, 2001). Exemplary transport vectors include cationized albumin or the OX26 monoclonal antibody to the transferrin receptor; these proteins undergo absorptive-mediated and receptor-mediated transcytosis through the BBB, respectively. Natural cell metabolites that can be used as targeting groups, include, inter alia, putrescine, spermidine, spermine, or DHA. Other exemplary targeting moieties include folate or biotin (see, e.g., U.S. Pat. No. 5,416,016); mannosides (Umezawa et al., Biochem. Biophys. Res. Commun. 153: 1038, 1988); antibodies (P. G. Bloeman, et al., FEBS Lett. 357: 140, 1995); M. Owais, et al., Antimicrob. Agents Chemother. 39: 180, 1995); surfactant protein A receptor (Briscoe et al., Am. J. Physiol. 1233: 134, 1995); gp120 (Schreier et al., J. Biol. Chem. 269: 9090, 1994; see also, K. Keinanen and M. L. Laukkanen, FEBS Lett. 346: 123, 1994; J. J. Killion and I. J. Fidler, Immunomethods 4: 273, 1994).

Examples of other BBB transport vectors that target receptor-mediated transport systems into the brain include factors such as insulin, insulin-like growth factors (“IGF-I,” and “IGF-II”), angiotensin II, atrial and brain natriuretic peptide (“ANP,” and “BNP”), interleukin I (“IL-1”) and transferrin. Monoclonal antibodies to the receptors that bind these factors can also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide can also cross the brain via absorptive-mediated transcytosis and are potential transport vectors.

Other BBB transport vectors target systems for transporting nutrients into the brain. Examples of such BBB transport vectors include hexose moieties, e.g., glucose and monocarboxylic acids, e.g., lactic acid and neutral amino acids, e.g., phenylalanine and amines, e.g., choline and basic amino acids, e.g., arginine, nucleosides, e.g., adenosine and purine bases, e.g., adenine, and thyroid hormone, e.g., triiodothyridine. Antibodies to the extracellular domain of nutrient transporters can also be used as transport vectors. Other possible vectors include angiotensin II and ANP, which can be involved in regulating BBB permeability.

In some cases, the bond linking the therapeutic agent to the transport vector can be cleaved following transport into the brain in order to liberate the biologically active agent. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, can also be used. Avidin itself can be a drug transport vector.

Transcytosis, including receptor-mediated transport of compositions across the blood brain barrier, can also be suitable for the agents of the invention. Transferrin receptor-mediated delivery is disclosed in U.S. Pat. Nos. 5,672,683; 5,383,988; 5,527,527; 5,977,307; and 6,015,555. Transferrin-mediated transport is also known. (P. M. Friden et al., Pharmacol. Exp. Ther. 278: 1491-98, 1996; H. J. Lee, J. Pharmacol. Exp. Ther. 292: 1048-52, 2000). EGF receptor-mediated delivery is disclosed in (Y. Deguchi et al., Bioconjug. Chem. 10: 32-37, 1999), and transcytosis is described in (A. Cerletti et al., J. Drug Target. 8: 435-46, 2000). Insulin fragments have also been used as carriers for delivery across the blood brain barrier. (M. Fukuta et al., Pharm. Res. 11: 1681-88, 1994). Delivery of agents via a conjugate of neutral avidin and cationized human albumin has also been described. (Y. S. Kang, et al., Pharm. Res. 1: 1257-64, 1994).

Other modifications in order to enhance penetration of the agents of the invention across the blood brain barrier can be accomplished using methods and derivatives known in the art. For example, U.S. Pat. No. 6,024,977 discloses covalent polar lipid conjugates for targeting to brain and central nervous system. U.S. Pat. No. 5,017,566 discloses cyclodextrin derivatives comprising inclusion complexes of lipoidal forms of dihydropyridine redox targeting moieties. U.S. Pat. No. 5,023,252 discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone or lactone. U.S. Pat. No. 5,024,998 discloses parenteral solutions of aqueous-insoluble drugs with cyclodextrin derivatives. U.S. Pat. No. 5,039,794 discloses the use of a metastatic tumor-derived egress factor for facilitating the transport of compounds across the blood-brain barrier. U.S. Pat. No. 5,112,863 discloses the use of N-acyl amino acid derivatives as antipsychotic drugs for delivery across the blood-brain barrier. U.S. Pat. No. 5,124,146 discloses a method for delivery of therapeutic agents across the blood-brain barrier at sites of increase permeability associated with brain lesions. U.S. Pat. No. 5,153,179 discloses acylated glycerol and derivatives for use in a medicament for improved penetration of cell membranes. U.S. Pat. No. 5,177,064 discloses the use of lipoidal phosphonate derivatives of nucleoside antiviral agents for delivery across the blood-brain barrier. U.S. Pat. No. 5,254,342 discloses receptor-mediated transcytosis of the blood-brain barrier using the transferrin receptor in combination with pharmaceutical compounds that enhance or accelerate this process. U.S. Pat. No. 5,258,402 discloses treatment of epilepsy with imidate derivatives of anticonvulsive sulfamate. U.S. Pat. No. 5,270,312 discloses substituted piperazines as central nervous system agents. U.S. Pat. No. 5,284,876 discloses fatty acid conjugates of dopamine drugs. U.S. Pat..No. 5,389,623 discloses the use of lipid dihydropyridine derivatives of anti-inflammatory steroids or steroid sex hormones for delivery across the blood-brain barrier. U.S. Pat. No. 5,405,834 discloses prodrug derivatives of thyrotropin releasing hormone. U.S. Pat. No. 5,413,996 discloses acyloxyalkyl phosphonate conjugates of neurologically-active drugs for anionic sequestration of such drugs in brain tissue. U.S. Pat. No. 5,434,137 discloses methods for the selective opening of abnormal brain tissue capillaries using bradykinin infused into the carotid artery. U.S. Pat. No. 5,442,043 discloses a peptide conjugate between a peptide having a biological activity and incapable of crossing the blood-brain barrier and a peptide which exhibits no biological activity and is capable of passing the blood-brain barrier by receptor-mediated endocytosis. U.S. Pat. No. 5,466,683 discloses water soluble analogues of an anticonvulsant for the treatment of epilepsy. U.S. Pat. No. 5,525,727 discloses compositions for differential uptake and retention in brain tissue comprising a conjugate of a narcotic analgesic and agonists and antagonists thereof with a lipid form of dihydropyridine that forms a redox salt upon uptake across the blood-brain barrier that prevents partitioning back to the systemic circulation.

Still further examples of modifications that enhance penetration of the blood brain barrier are described in International (PCT) Application Publication Number WO 85/02342, which discloses a drug composition comprising a glycerolipid or derivative thereof. PCT Publication Number WO 089/11299 discloses a chemical conjugate of an antibody with an enzyme which is delivered specifically to a brain lesion site for activating a separately-administered neurologically-active prodrug. PCT Publication Number WO 91/04014 discloses methods for delivering therapeutic and diagnostic agents across the blood-brain barrier by encapsulating the drugs in liposomes targeted to brain tissue using transport-specific receptor ligands or antibodies. PCT Publication Number WO 91/04745 discloses transport across the blood-brain barrier using cell adhesion molecules and fragments thereof to increase the permeability of tight junctions in vascular endothelium. PCT Publication Number WO 91/14438 discloses the use of a modified, chimeric monoclonal antibody for facilitating transport of substances across the blood-brain barrier. PCT Publication Number WO 94/01131 discloses lipidized proteins, including antibodies. PCT Publication Number WO 94/03424 discloses the use of amino acid derivatives as drug conjugates for facilitating transport across the blood-brain barrier. PCT Publication Number WO 94/06450 discloses conjugates of neurologically-active drugs with a dihydropyridine-type redox targeting moiety and comprising an amino acid linkage and an aliphatic residue. PCT Publication Number WO 94/02178 discloses antibody-targeted liposomes for delivery across the blood-brain barrier. PCT Publication Number WO 95/07092 discloses the use of drug-growth factor conjugates for delivering drugs across the blood-brain barrier. PCT Publication Number WO 96/00537 discloses polymeric microspheres as injectable drug-delivery vehicles for delivering bioactive agents to sites within the central nervous system. PCT Publication Number WO 96/04001 discloses omega-3-fatty acid conjugates of neurologically-active drugs for brain tissue delivery. PCT WO 96/22303 discloses fatty acid and glycerolipid conjugates of neurologically-active drugs for brain tissue delivery.

In general, it is well within the ordinary skill in the art to prepare an ester, amide or hydrazide derivative of an agent of the invention, for example, from the corresponding carboxylic acid and a suitable reagent. For instance, a carboxylic acid-containing compound, or a reactive equivalent thereof, can be reacted with a hydroxyl-containing compound, or a reactive equivalent thereof, so as to provide the corresponding ester. (See, e.g., COMPREHENSIVE ORGANIC TRANSFORMATIONS, 2^{nd} Ed., by R. C. Larock, VCH Publishers John Wiley & Sons, Ltd., 1989; MARCH's ADVANCED ORGANIC CHEMISTRY, 5^{th} Ed., by M. B. Smith and J. March, John Wiley & Sons, Ltd. (2000).

12. Labels

The particular label or detectable group used in the assay is not a critical aspect of the invention, so long as it does not significantly interfere with the specific binding of the small molecule chemical inhibitors or siRNA inhibitors of PGE2 activity, e.g., EP2 activity, ligand mimetics, derivatives and analogs thereof, antibodies, or nucleic acid compositions, e.g., antisense oligonucleotides or double stranded RNA oligonucleotides (RNAi), used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹ I, ¹¹²In, ⁹⁹mTc), other imaging agents such as microbubbles (for ultrasound imaging), ¹⁸F, ¹¹C, ¹⁵O, (for Positron emission tomography), ^{99m}TC, ¹¹¹ In (for Single photon emission tomography), enzymes (e.g., horse radish peroxidase, alkaline phosphatase and others commonly used in an ELISA), and calorimetric labels such as colloidal gold or colored glass or plastic (e.g. polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene Oreg.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, and the like Chemiluminescent compounds include luciferin, and 2,3-dihydrophthalazinediones, e.g., luminol. For a review of various labeling or signal producing systems which can be used, see, U.S. Pat. No. 4,391,904, incorporated herein by reference in its entirety and for all purposes.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple calorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Frequently, the PGE2 polypeptide, e.g., EP2 polypeptides will be labeled by joining, either covalently or non-covalently, a substance which provides for a detectable signal.

13. Pharmaceutical Preparations

In another embodiment, the present invention relates to pharmaceutical compositions comprising agents as disclosed herein for the treatment of neurodegenerative diseases, such as an amyloid-β or related disease, as well as methods of manufacturing such pharmaceutical compositions.

In general, the agents of the present invention can be obtained by the methods illustrated, for example, in the patents and patent applications referred to herein, or by modifications thereof, using readily available starting materials, reagents and conventional synthesis procedures. In these reactions, it is also possible to make use of variants which are in themselves known, but are not mentioned here. Functional and structural equivalents of the agents described herein and which have the same general properties, wherein one or more simple variations of substituents are made which do not adversely affect the essential nature or the utility of the agent.

The agents of the invention can be supplied in a solution with an appropriate solvent or in a solvent-free form (e.g., lyophilized). In another aspect of the invention, the agents and buffers necessary for carrying out the methods of the invention can be packaged as a kit. The kit can be commercially used according to the methods described herein and can include instructions for use in a method of the invention. Additional kit components can include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

A. Treatment Regimes

The invention provides pharmaceutical compositions comprising one or a combination of small molecule chemical inhibitors, or siRNA inhibitors, or dominant-negative mutants of PGE2, e.g., EP2 activity, (monoclonal, polyclonal or single chain Fv; intact or binding fragments thereof) or nucleic acid compositions, e.g., antisense oligonucleotides, double stranded RNA oligonucleotides (RNAi) or DNA oligonucleotides (vectors) containing nucleotide sequences encoding for the transcription of shRNA molecules, formulated together with a pharmaceutically acceptable carrier. Some compositions include a combination of multiple (e.g., two or more) small chemical molecules, siRNA molecules, monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

In prophylactic applications, pharmaceutical compositions or medicaments are administered to a patient susceptible to, or otherwise at risk of a neurodegenerative disease or condition (i.e., AD or PD) in an amount sufficient to eliminate or reduce the risk, lessen the severity, or delay the outset of the disease, including biochemical, histologic and/or behavioral symptoms of the neurodegenerative disease, its complications and intermediate pathological phenotypes presenting during development of the disease. In therapeutic applications, compositions or medicants are administered to a patient suspected of, or already suffering from such a neurodegenerative disease in an amount sufficient to cure, or at least partially arrest, the symptoms of the neurodegenerative disease (biochemical, histologic and/or behavioral), including its complications and intermediate pathological phenotypes in development of the neurodegenerative disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if the response starts to wane.

B. Effective Dosages

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic agent calculated to produce the desired therapeutic effect in association with the required pharmaceutical vehicle. The specification for the dosage unit forms of the invention are dictated by and directly dependent on (a) the unique characteristics of the therapeutic agent and the particular therapeutic effect to be achieved, and (b) the limitations inherent in the art of compounding such a therapeutic agent for the treatment of, for example, amyloid or synuclein deposition in subjects.

Effective doses of the small molecule chemical inhibitors, siRNA inhibitors, or dominant-negative mutants of PGE2 activity, e.g., EP2 activity, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of neurodegenerative disease described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with a small chemical molecule, nucleic acid, siRNA, or antibody composition, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more small chemical molecules or siRNA molecules with different binding specificities are administered simultaneously, in which case the dosage of each small chemical molecule, siRNA molecule, or antibody administered falls within the ranges indicated. Small chemical molecule, siRNA molecule, or antibody is usually administered on multiple occasions. Intervals between single dosages can be weekly, monthly or yearly. Intervals can also be irregular as indicated by measuring blood levels of small chemical molecule, siRNA molecule, or antibody in the patient. In some methods, dosage is adjusted to achieve an antibody concentration of 1-1000 μg/mi and in some methods 25-300 μg/ml. Alternatively, antibody can be administered as a sustained release formulation, in which case less frequent administration is required. Dosage and frequency vary depending on the half-life of the compound in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for small chemical molecules, siRNA molecules, or nucleic acids range from about 10 ng to 1 g, 100 ng to 100 mg, 1 μg to 10 mg, or 30-300 μg DNA per patient. Doses for infectious viral vectors vary from 10-100, or more, virions per dose.

C. Routes of Administration

Small chemical molecule, siRNA molecule, or antibody compositions for treatment or amelioration of neurodegenerative diseases or disorders, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of neurodegenerative diseases or disorders, e.g., AD or PD, can be administered by parenteral, topical, intravenous, oral, subcutaneous, intraarterial, intracranial, intraspinal, intraperitoneal, intranasal or intramuscular means for prophylactic as inhalants for small chemical molecule, siRNA molecule or antibody preparations targeting neurodegenerative diseases or disorders, and/or therapeutic treatment. The most typical route of administration of an agent is subcutaneous although other routes can be equally effective. The next most common route is intramuscular injection. This type of injection is most typically performed in the arm or leg muscles. In some methods, particular therapeutic antibodies are delivered directly into the cranium. In some methods, antibodies are administered as a sustained release composition or device, such as a Medipad™ device.

Agents of the invention can optionally be administered in combination with other agents that are at least partly effective in treating various neurodenerative diseases including various AD and PD. Agents of the invention can also be administered in conjunction with other agents that increase passage of the agents of the invention across the blood-brain barrier (BBB) (discussed supra).

D. Formulations

Small chemical molecule, siRNA molecule, or antibody inhibitors of PGE2, for example EP2, nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of neurodegenerative diseases and disorders, are often administered as pharmaceutical compositions comprising an active therapeutic agent, i.e., and a variety of other pharmaceutically acceptable components. (See REMINGTON: THE SCIENCE AND PRACTICE OF PHARMACY, 20^{th} Ed., Gennaro, A. R. (ed.), Mack Publishing Company, Easton, Pa., 2000). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. (Langer, Science 249: 1527, 1990 and Hanes, Advanced Drug Delivery Reviews 28: 97-119, 1997). The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications discussed further below.

Dispersions can be prepared in glycerol, liquid polyethylene glycols, and mixtures thereof and in oils. Under ordinary conditions of storage and use, these preparations can contain a preservative to prevent the growth of microorganisms.

To administer the therapeutic agent by other than parenteral administration, it can be necessary to coat the agent with, or co-administer the agent with, a material to prevent its inactivation. For example, the therapeutic agent can be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7: 27, 1984).

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. (Glenn et al., Nature 391: 851, 1998). Co-administration can be achieved by using the components as a mixture or as linked molecules obtained by chemical crosslinking or expression as a fusion protein.

The pharmaceutical compositions are generally formulated as sterile, substantially isotonic and in full compliance with all Good Manufacturing Practice (GMP) regulations of the U.S. Food and Drug Administration.

Suitable pharmaceutically acceptable carriers include, without limitation, any non-immunogenic pharmaceutical adjuvants suitable for oral, parenteral, nasal, mucosal, transdermal, intravascular (IV), intraarterial (IA), intramuscular (IM), and subcutaneous (SC) administration routes, such as phosphate buffer saline (PBS).

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic agent in the required amount in an appropriate solvent with one or a combination of ingredients enumerated above, as required, followed by filtered sterilization. Generally, dispersions are prepared by incorporating the therapeutic agent into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic agent) plus any additional desired ingredient from a previously sterile-filtered solution thereof.

The therapeutic agent can be orally administered, for example, with an inert diluent or an assimilable edible carrier. The therapeutic agent and other ingredients can also be enclosed in a hard or soft shell gelatin capsule, compressed into tablets, or incorporated directly into the subject's diet. For oral therapeutic administration, the therapeutic agent can be incorporated with excipients and used in the form of ingestible tablets, buccal tablets, troches, capsules, elixirs, suspensions, syrups, wafers, and the like. The percentage of the therapeutic agent in the compositions and preparations can, of course, be varied. The amount of the therapeutic agent in such therapeutically useful compositions is such that a suitable dosage will be obtained.

The present invention therefore includes pharmaceutical formulations comprising the agents obtained by the methods described herein, including pharmaceutically acceptable salts thereof, in pharmaceutically acceptable carriers for aerosol, oral and parenteral administration. Also, the present invention includes such agents, or salts thereof, which have been lyophilized and which can be reconstituted to form pharmaceutically acceptable formulations for administration, as by intravenous, intramuscular, or subcutaneous injection. Administration can also be intradermal or transdermal.

In accordance with the present invention, an agent obtained by the methods described herein, and pharmaceutically acceptable salts thereof, can be administered orally or through inhalation as a solid, or can be administered intramuscularly or intravenously as a solution, suspension or emulsion. Alternatively, the agents or salts can also be administered by inhalation, intravenously or intramuscularly as a liposomal suspension.

Pharmaceutical formulations are also provided which are suitable for administration as an aerosol, by inhalation. These formulations comprise a solution or suspension of the desired agent of the invention, or a salt thereof, or a plurality of solid particles of the agent or salt. The desired formulation can be placed in a small chamber and nebulized. Nebulization can be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing the solid agent obtained by the methods, or a salt thereof, in any appropriate manner known in the art, such as by micronization. The size of the solid particles or droplets will be, for example, from about 1 to about 2 microns. In this respect, commercial nebulizers are available to achieve this purpose.

A pharmaceutical formulation suitable for administration as an aerosol can be in the form of a liquid, the formulation will comprise a water-soluble agent obtained by the methods, or a salt thereof, in a carrier which comprises water. A surfactant can be present which lowers the surface tension of the formulation sufficiently to result in the formation of droplets within the desired size range when subjected to nebulization.

Peroral compositions also include liquid solutions, emulsions, suspensions, and the like. The pharmaceutically acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol. and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions can also contain one or more components such as sweeteners, flavoring agents and colorants disclosed above.

Pharmaceutical compositions can also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Other compositions useful for attaining systemic delivery of the subject agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above can also be included.

The compositions of this invention can also be administered topically to a subject, e.g., by the direct laying on or spreading of the composition on the epidermal or epithelial tissue of the subject, or transdermally via a skin “patch” or using transferosomes. (See, e.g., Paul et al., Eur. J. Immunol. 25: 3521-24, 1995; Cevc et al., Biochem. Biophys. Acta 1368: 201-15, 1998). Such compositions include, for example, lotions, creams, solutions, gels and solids. These topical compositions can comprise an effective amount, usually at least about 0.1%, or evan from about 1% to about 5%, of an agent of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier can include pharmaceutically acceptable emolients, emulsifiers, thickening agents, solvents and the like.

Active agents are administered at a therapeutically effective dosage sufficient to inhibit amyloid deposition in a subject. A “therapeutically effective” dosage inhibits amyloid or synuclein deposition by a statistically significant amount relative to untreated subjects. In another embodiment, a therapeutically effective dosage inhbitis amyloid or synuclein deposition by, for example, at least about 20%, or by at least about 40%, or even by at least about 60%, or by at least 80%. In the case of an Alzheimer's or Parkinson's subject, a “therapeutically effective” dosage stabilizes cognitive function or prevents a further decrease in cognitive function (i.e., preventing, slowing, or stopping disease progression). The present invention accordingly provides therapeutic drugs. By “therapeutic” or “drug” is meant an agent having a beneficial ameliorative or prophylactic effect on a specific disease or condition in a living human or non-human animal.

Furthermore, active agents are administered at a therapeutically effective dosage sufficient to decrease deposition in a subject of amyloid protein, e.g., Aβ40 or Aβ42, or synuclein protein, e.g., α-synuclein. A therapeutically effective dosage inhibits amyloid deposition by, for example, at least about 15%, or by at least about 40%, or even by at least 60%, or at least by about 80% relative to untreated subjects.

In another embodiment, active agents are administered at a therapeutically effective dosage sufficient to increase or enhance amyloid protein, e.g., Aβ40 or Aβ42, or synuclein protein, e.g., β-synuclein, in the blood of a subject. A therapeutically effective dosage increases the concentration by a statistically significant amount relative to untreated subjects.

In yet another embodiment, active agents are administered at a therapeutically effective dosage sufficient to improve ADAS-cog test scores by, e.g., at least about 1 point, at least about 2 points, at least about 3 points, at least about 4 points, at least about 5 points, at least about 10 points, at least about 12 points, at least about 15 points, or at least about 20 points relative to untreated subjects.

E. Toxicity

Preferably, a therapeutically effective dose of the small chemical molecule, siRNA molecule, antibody, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population), the LD₁₀₀ (the dose lethal to 100% of the population), or the ED₅₀ (the dose therapeutically effective in 50% of the population). The dose ratio between toxic and therapeutic effects is the therapeutic index and can be expressed as the ratio LD₅₀/ED₅₀, and usually a larger therapeutic index are more efficacious. While agents that exhibit toxic side effects can be used, care should be taken to design a delivery system that targets such agents to the site of affected tissue in order to minimize potential damage to uninfected cells and, thereby, reduce side effects.

The data obtained from these cell culture assays and animal studies can be used in formulating a dosage range that is not toxic for use in human. The dosage of the agents described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1)

It is understood that appropriate doses depend upon a number of factors within the knowledge and experience of the ordinarily skilled physician, veterinarian, or researcher. The dose(s) of the small molecule or other agents will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency with respect to the expression or activity to be modulated. Such appropriate doses can be determined using the assays described herein. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

F. Pharmaceutically Acceptable Salts

Certain embodiments of the present agents can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agents of the present invention. These salts can be prepared in situ during the final isolation and purification of the agents of the invention, or by separately reacting a purified agent of the invention in its free base form with a suitable organic or inorganic acid, and isolating the salt thus formed.

Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethanesulfonate, and laurylsulphonate salts and the like. (See, e.g., Berge et al., J. Pharm. Sci. 66: 1-19, 1977).

In other cases, the agents of the present invention can contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention.

These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolaamine, piperazine and the like.

Those skilled in the art will recognize, or be able to ascertain using no more than routine experimentation, many equivalents to the specific embodiments and methods described herein. Such equivalents are intended to be encompassed by the scope of the following claims. All patents, patent applications, and literature references cited herein are hereby expressly incorporated by reference in their entirety. This invention is further illustrated by the following examples which should not be construed as limiting.

“Pharmaceutically acceptable salts” also includes, for example, derivatives of agents modified by making acid or base salts thereof, as described further below and elsewhere in the present application. Examples of pharmaceutically acceptable salts include mineral or organic acid salts of basic residues such as amines; and alkali or organic salts of acidic residues such as carboxylic acids. Pharmaceutically acceptable salts include the conventional non-toxic salts or the quaternary ammonium salts of the parent agent formed, for example, from non-toxic inorganic or organic acids. Such conventional non-toxic salts include those derived from inorganic acids such as hydrochloric, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid. Pharmaceutically acceptable salts can be synthesized from the parent agent which contains a basic or acidic moiety by conventional chemical methods. Generally, such salts can be prepared by reacting the free acid or base forms of these agents with a stoichiometric amount of the appropriate base or acid in water or in an organic solvent, or in a mixture of the two.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values in between these values and ranges can also be the upper or lower limits of a range.

The invention will be further described with reference to the following examples; however, it is to be understood that the invention is not limited to such examples.

G. Kits

Also within the scope of the invention are kits comprising the small chemical molecule, dominant-negative molecule, siRNA molecule, antibody, or nucleic acid compositions (e.g., antisense oligonucleotides, double stranded RNA oligonucleotides (RNAi), or DNA oligonucleotides (vectors) containing nucleotide sequences encoding for the transcription of shRNA molecules) of the invention and instructions for use. The kit can further contain a least one additional reagent, or one or more additional human antibodies of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

The invention will be further described with reference to the following examples; however, it is to be understood that the invention is not limited to such examples.

### EXEMPLARY EMBODIMENTS

Methods

Animals and materials. Mice homozygous for disruption of the gene that encodes the EP2 were backcrossed >12 generations to the Balb/c genetic background. Balb/c wild-type (wt) control mice were obtained from Charles River Laboratories (Wilmington, Mass.). Mice were maintained in a temperature-controlled specific pathogen-free facility with a strict 12-hour light/dark cycle and with free access to food and water. Antibodies 4G8 and 6E10 (specific for Aβ₁₋₁₇ and so does not recognize the p3 fragment of APP) were from Signet Laboratories (Dedham, Mass.). CD11b was from Serotec (Raleigh, N.C.). Antibodies against microtubule-associated protein 2 (MAP-2) and neuronal nuclei (NeuN) were from Chemicon (Temecula, Calif.). Alexa fluorescent-labeled secondary antibodies were from Molecular Probes (Eugene, Oreg.). 4,6-diamidino-2-phenylindole (DAPI) containing mounting medium was from Vector Laboratories (Burlingame, Calif.). Poly-D-lysine was from BD Biosciences (Bedford, Mass.). Papain and DNase I were from Worthington Biochemical (Lakewood, N.J.). Synthetic Aβ₁₋₄₂ was from Bachem (Torrance, Calif.) and fluorescein-labeled Aβ₁₋₄₂ was from rPeptide (Athens, Ga.). AH6809, butaprost, and 17-phenyl trinor prostaglandin E2 (PTPE2) were from Cayman (Ann Arbor, Mich.). SC51089 was from Biomol (Plymouth Meeting, Pa.). Forskolin and bisindolylmaleimide (BIM) was from Calbiochem (La Jolla, Calif.). Culture media, heat-inactivated fetal bovine serum (FBS), bovine calf serum (BCS), and penicillin/streptomycin were from Invitrogen (Carlsbad, Calif.). Other chemicals were purchased from Sigma-Aldrich (St. Louis, Mo.) unless stated otherwise.

Ex vivo studies. Frozen AD hippocampus was sliced into 10 μm thick sections using cryostat and sections were mounted onto poly-D-lysine coated cover slip. Sections were stored at −80° C. and, to minimize the variation among sections used for wt and EP2^{-/-} microglia, consecutive sections were selected. Primary microglia cultures were derived from cortices of P1-3 neonates. Cells were dissociated using enzyme solution containing DMEM, EDTA (0.5 mM), L-cysteine (0.2 mg/ml), Papain (15 /ml), Dnase I (200 μg/ml) followed by trituration. Culture medium (DMEM with 10% FBS, 100 units/ml penicillin, and 100 μg/ml streptomycin) was changed after 24 hr initial seeding. Microglia at 14th day in vitro (DIV) were separated from the underlying astrocytic monolayer by gentle agitation using their differential adhesive properties and were seeded on hippocampal sections at 1×10⁵ cells per section in microglial culture medium for 2 hr followed by additional 48 hr incubation in serum-free DMEM containing penicillin and streptomycin. To determine purity of microglia, 1×10⁴ cells were cultured on chambered slide with treatment of 100 nM of aggregated Aβ₁₋₄₂ for 24 hr followed by cytochemistry analysis using a microglial marker, CD11b. Percent of positive microglia was normalized by DAPI nuclei counterstaining. From six independent experiments, purity of microglia was 99.4+0.2%. For immunohistochemical analysis, cultures were fixed with 4% paraformaldehyde in PBS and subjected to formic acid (88%) treatment prior to application of antibodies against CD11b (1:50) and Aβ (4G8, 1:200). Mounting medium containing DAPI was used to label nuclei. Images were randomly selected using Leica confocal/two-photon imaging system. A representative image with comparable number of microglia between wt and EP2^{-/-} was compared for reduction of Aβ plaques. All images were subjected to quantification of CD11b using MetaMorph computer assisted software. Data were presented as average positive area in arbitrary units per microglia on top of human brain sections. For Western blot analysis, cultures were lysed with 8M urea containing proteinase inhibitor cocktail. Lysates with equal amount of protein were subjected to 16.5% Tris-Tricine SDS PAGE followed by Western blot using 6E10 antibody. ECL was used to reveal Aβ species and intensity of the corresponding bands were quantified using Bio-Rad GS-710 Imaging Densitometer (Hercules, Calif.). Six separated experiments were performed for each analysis.

Microglial Aβ phagocytosis. At DIV 14, microglia were seeded in 6-well plates at 1.5×10⁵ cells per well in microglial culture medium. After 2hr seeding, medium was changed with 1.5 ml of serum-free DMEM medium containing penicillin and streptomycin. To make aggregated Aβ for phagocytosis, Aβ was dissolved in 2 mM NaOH and was diluted into 10 μM of stock solution (100×) in PBS followed by incubation at 37° C. for 2 hr and monitored by turbidity. For studying signaling pathways involved in Aβ phagocytosis, some microglia were pre-treated for 1 hr with AH6809 (20 μM), butaprost (1-50 EM), SC51089 (10 μM), PTPE2 (20 μM), forskolin (50 μM), or BIM (10 μM) prior to treatment with aggregated fluorescein labeled AP₁₋₄₂ (100 nM). After 24hr incubation of Aβ, cells were washed with PBS followed by trypsinization (0.05%) and centrifugation. Cells were then re-suspended in PBS and subjected to flow cytometry. Flow cytometer (FACScan, BD Biosciences, San Jose, Calif.) set at un-gated 10,000 cells per measure was used to analyze Aβ uptake as an indication of Aβ phagocytosis. Data were analyzed using Summit Mo-Flo software (Cytomation, Fort Collins, Colo.). Phagocytosis was presented as percent of mean FITC intensity in wt controls treated with Aβ. Some cells were plated onto chambered slides at 1×10⁴ for assessing cellular uptake of Aβ using fluorescent microscope (Nikon TE200).

Aβ activated microglia-mediated neurotoxicity. For primary neuronal cultures, embryonic (E16-17) cortices from wt mice were extracted, partially digested with proteases, and gently disrupted prior to plating at 5×10⁵ cells per well in poly-D-lysine/laminin (BD Biosciences) coated 24-well plates. One hr after seeding, medium was replaced with neurobasal medium with B-27 supplement, penicillin (100 units/ml), streptomycin (100 μg/ml), and glutamine (2 mM). Cytosine arabinoside (2.5 μM) was added on the second day. Purity for neuronal cultures was 98±1% as measured by NeuN antibody. DIV 14 primary microglia derived from wt and EP₂^{-/-} mice were plated at 1×10⁵ per well of wt neurons (DIV 6). To make aggregated Aβ for neurotoxicity assay, 1 mg of Aβ₁₋₄₂ was dissolved in 20 μl of DMSO and was then diluted into PBS to make 500 μM stock solution followed by 37° C. incubation for 24 hr. Freshly prepared pre-aggregated Aβ at 12 μM was added into co-culture medium containing DMEM, 10% FBS, 100 units/ml penicillin, and 100 μg/ml streptomycin for 24 hr. Cultures were washed twice with PBS and subjected to fixation with 4% paraformaldehyde in PBS at 4° C. for 1 hr. Terminal deoxynucleotidyl transferase-mediated deoxyuridinetripohsphate end-labeling (TUNEL) assay (Roche Applied Science, Indianapolis, Ind.) was performed per manufacturer's instruction followed by fluorescent immunocytochemistry using MAP-2 antibody to identify neurons. Neurotoxicity was presented as percentage of co-positivity for both TUNEL and MAP-2 in all MAP-2 positive neurons.

Cell count and levels of secreted cytokines. To measure cell proliferation, microglia from wt and EP2^{-/-} were initially seeded at 1×10⁵ cells per 24-well culture plate in serum containing medium. After 2 hr of incubation, some cultures were changed to serum free conditions. After additional 24 hr of incubation, cells were washed with PBS and were trypsinized followed by cell count. Six separate experiments were performed to obtain cell count. For cytokine analysis, culture medium from serum free condition was collected from wt and EP₂^{-/-} microglial cultures and were stored at −20° C. until measurements were performed. Cytokine/chemokine membrane array (Ray Biotech, Norcross, Ga.) were used to screen for 32 molecules: interleukin (IL)-2, IL-3, IL-4, IL-5, IL-6, IL-9, IL-10, IL12, IL-12p70, IL-13, IL-17, interferon (IFN)-□, six conserved cytokines (6Ckine), cutaneous T cell-attracting chemokine (CTACK), eotaxin, granulocyte colony-stimulating factor (GCSF), granulocyte macrophage colony stimulating factor (GM-CSF), cytokine-induced neutrophil chemoattractant (KC), leptin, macrophage chemoattractant protein (MCP)-1, MCP-5, macrophage inflammatory protein (MIP)-1α, MIP-2, MIP-3β, RANTES (regulated upon activation, normal T cell expressed and secreted), stem cell factor (SCF), thymus and activation regulated chemokine (TARC), tissue inhibitors of matrix metalloproteinases (TIMP)-1, tumor necrosis factor (TNF)-α, soluble TNF receptor type I (sTNFrI), thrombopoietin (TPO), and vascular endothelial cell growth factor (VEGF). ELISA kits for mouse MCP-1 (Pierce, Rockford, Ill.), MIP-1α (Biosource International, Camarillo, Calif.), and M-CSF (Pierce, Rockford, Ill.) were used according to manufacturers' instructions. Levels of cytokines were obtained at pg per ml of culture medium from 10⁵ microglia cultivated at basal conditions.

Results

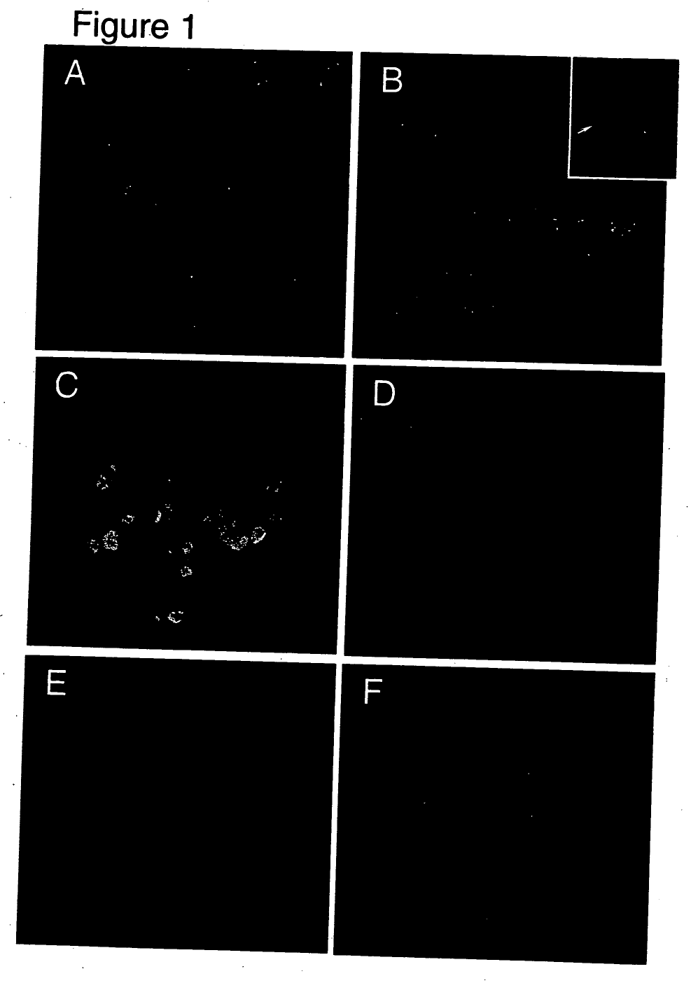

Enhanced phagocytosis of Aβ from senile plaques in human hippocampus by EP2^{-/-} microglia ex vivo. We first employed a human ex vivo model by incubating primary mouse microglia on top of hippocampal sections from patients who died with AD and participated in the rapid autopsy program at the University of Washington. Confocal images showed abundant Aβ-immmunoreactive (IR) material in the distribution of senile plaques in an AD tissue section that was incubated with wt microglia (FIG. 1A) and, similar to the results of others, no substantial change from a consecutive tissue section that was incubated without wt microglia (not shown). In contrast, incubation of the next tissue section with EP2^{-/-} microglia (FIG. 1B) markedly reduced Aβ-IR material in tissue. Although several possible mechanisms exist to explain the reduction of Aβ-IR material in human hippocampus caused by EP2^{-/-} microglia, EP2^{-/-} microglia displayed morphologic features of Aβ phagocytosis, including pseudopodia engulfing Aβ-IR material (arrow in inset of FIG. 1B), that were not present in wt microglia incubated with AD tissue. Moreover, EP2^{-/-} microglia incubated with AD tissue contained Aβ-IR material (FIG. 1D) inside phagocytic vacuoles as indicated by intracellular compartments that were IR for CD11b, an indicator for microglial activation (Walker et al., Neurobiol. Aging 22: 957-966; 2001). (FIG. 1E). In addition, DNA fragments, likely released from necrotic cells in the tissue section, were also seen within these vacuoles (FIG. 1F). Co-localization among Aβ, CD11b, and DNA fragments is shown in FIG. 1C.

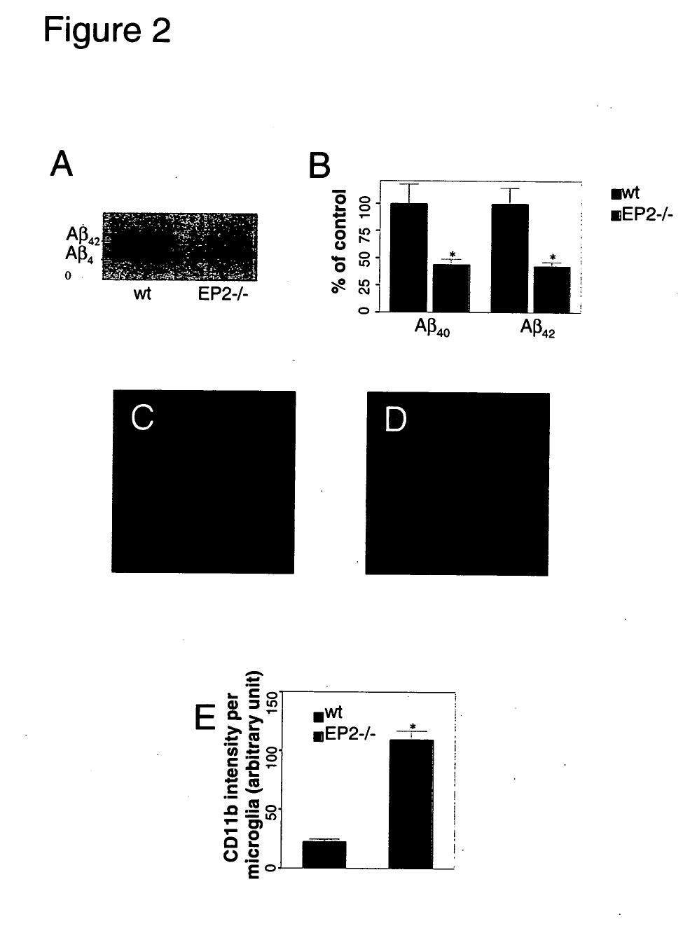

Microglial activation and reduction of Aβ₁₄₀ and Aβ₁₄₂ in human hippocampus by EP2^{-/-} microglia ex vivo. To quantify disappearance of Aβ peptides in this ex vivo model, samples were extracted and subjected to Western blot analysis. Aβ₁₋₄₀ and Aβ₁₋₄₂ levels from AD tissue sections were determined from the corresponding band density using densitometry. We found that, similar to the results of others, tissue incubated with wt microglia had Aβ1-40 and Aβ₁₋₄₂ levels that were no different from consecutive sections incubated without microglia (not shown). In contrast, tissue sections incubated with EP2^{-/-} microglia had Aβ₁₋₄₀ reduced to 44±5% (P<0.05, n=6) and Aβ₁₋₄₂ reduced to 43+4% (P<0.01, n=6) after 48 hr compared to consecutive tissue sections incubated with wt microglia (FIGS. 2A and 2B); by inspection, minimal clearance of Aβ immunoreactivity had occurred by 24 hr. Estimation of CD11b IR per microglial cell incubated with human brain tissue slices was performed using confocal images from wt (FIG. 2C) and EP2^{-/-} (FIG. 2D). Data showed that CD11b IR in wt or EP2^{-/-} microglia was 23±2 arbitrary fluorescence units (n=5) or 110±7 arbitrary fluorescence units (n=10, P<0.01), respectively (FIG. 2E); these data associate reduction of Aβ burden in AD brain section with a standard measure of microglial activation. Although the total number of microglia present on AD tissue were equivalent regardless of genotype at the end of incubation (159±17, n=5, wt microglia in 250× fields and 165±22, n=10, EP2^{-/-} microglia they were not similarly distributed; EP2^{-/-} microglia tended to aggregate around Aβ-IR plaques while wt microglia were more evenly dispersed across the tissue section, perhaps suggesting enhanced chemoattaction in EP2^{-/-} microglia. In combination, these observations showed that EP2^{-/-} microglia had enhanced phagocytic activity toward fibrillar Aβ peptides and reduced Aβ peptide tissue burden in human hippocampal slices much more effectively than wt microglia. Although the precise mechanisms of reduced Aβ tissue in these experiments have not been determined, we believe, as have most others (D'Andrea et al., Neurobiol Aging 25: 675-683, 2004; Rogers et al. Glia 40: 260-269, 2002; Das et al., J. Neurosci. 23: 8532-8538, 2003) that this occurs by degradation of phagocytosed protein.

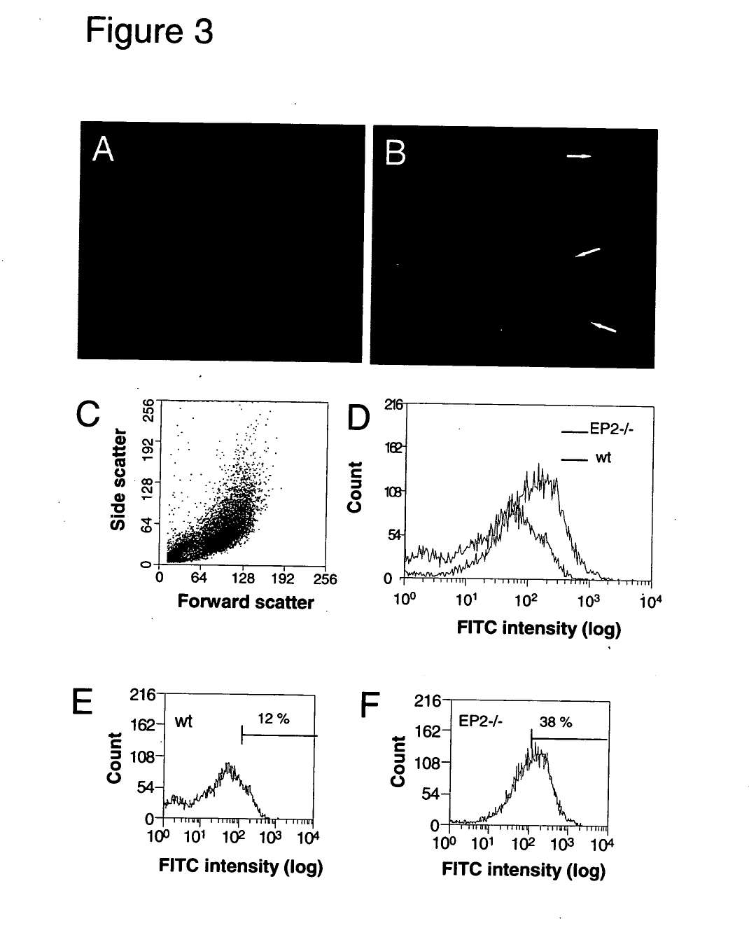

Microglial phagocytosis of aggregated fluorescein-labeled Aβ₁₋₄₂. To characterize better the enhanced Aβ₁₋₄₂ phagocytosis by EP2^{-/-} microglia, primary cultures of microglia from wt and EP2^{-/-} mice were incubated with aggregated Aβ₁₋₄₂ labeled with fluorescein and analyzed by microscopy and flow cytometry. Consonant with our ex vivo studies described above, EP2^{-/-} microglia (FIG. 3B) had greater Aβ₁₋₄₂ uptake compared to wt (FIG. 3A). Some EP2^{-/-} microglia displayed phagocytosed AO that exceeded that observed in wt microglia (arrow in FIG. 3B), while other EP2^{-/-} microglia appeared similar to their wt counterparts, suggesting a heterogeneous population of EP2^{-/-} microglia with a subpopulation of cells with enhanced phagocytic properties for Aβ₁₋₄₂. A representative scatter plot from flow cytometric analysis showed that granularity and size were not different between wt and EP2^{-/-} microglia (FIG. 3C). Flow cytometric data (FIG. 3D) showed a shift toward higher fluorescent intensity in EP2^{-/-} microglia compared to wt following incubation with fluorescein-labeled Aβ₁₋₄₂. Using a cut-off of the peak value for EP2^{-/-} microglia, the percent of tested cells with high fluorescent intensity in wt (FIG. 3E) or EP2^{-/-} microglia (FIG. 3F) was 12±1% (n=10) or 38±1% (n=10), respectively (P<0.001). However, there was overlap between microglia of the two genotypes at low Aβ₁₋₄₂ uptake (FIG. 3D). These data support the hypothesis that a subpopulation of EP2^{-/-} microglia has enhanced phagocytic properties for Aβ₁₋₄₂ and suggest that ablation of EP2 relieves an inhibitory mechanism thereby leading to a greater proportion of cells with heightened phagocytic activity for Aβ₁₋₄₂.

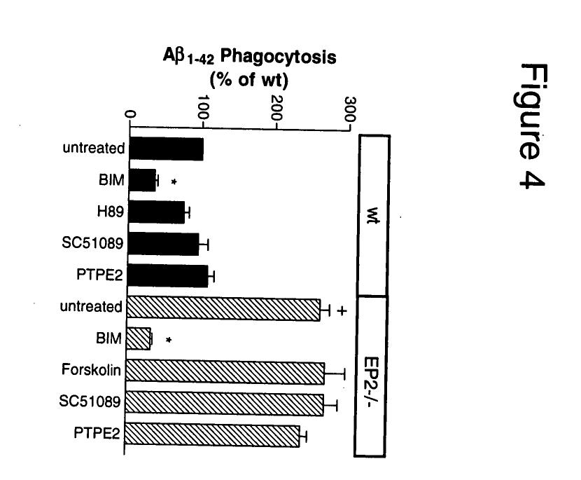

Signaling pathways for enhanced Aβ1-42 phagocytosis in EP2^{-/-} microglia. We next investigated the signaling events that underlie augmented Aβ₁₋₄₂ phagocytic activity in EP2^{-/-} microglia using the same flow cytometric technique (FIG. 4). Phagocytosis of Aβ₁₋₄₂ was 2.5- to 3-fold greater in EP₂^{-/-} microglia compared to wt (P<0.0001). Wt and EP₂^{-/-} microglia had 36±4% (p<0.001) and 35±4% (p<0.001) of wt basal Aβ₁₋₄₂ uptake when incubated with the PKC inhibitor BIM at 10 μM, suggesting that all of the enhanced Aβ₁₋₄₂ phagocytosis observed in EP2^{-/-} microglia and part of basal Aβ₁₋₄₂ phagocytosis in wt microglia are PKC-dependent. A PKA inhibitor (H89) was used in wt microglia and a PKA activator (forskolin) was used in EP2^{-/-} microglia to determine if this signaling pathway was contributing to the enhanced Aβ₁₋₄₂ uptake. H89 did not enhanced Aβ₁₋₄₂ uptake in wt microglia and so did not replicate the EP2^{-/-} phenotype, and forskolin did not reverse the enhanced Aβ₁₋₄₂ phagocytosis observed in EP2^{-/-} microglia. We next investigated the effects of the relatively selective EP1 antagonist, SC51089 at 10 μM, or EP1 agonist, PTPE2 at 20 μM, on Aβ₁₋₄₂ phagocytosis by microglia. We found that both drugs were without effect in either wt or EP2^{-/-} microglia, suggesting that EP1 activation or blockade does not significantly alter microglial Aβ₁₋₄₂ phagocytosis. Finally, butaprost, a relatively selective EP₂ agonist, did not alter Aβ₁₋₄₂ uptake in wt microglia (98±3% at 1 μM, 104±2% at 10 μM, and 92±2% at 50 μM, n=6 for each condition), suggesting that stimulation of EP2 does not have the opposing effect of ablating EP2. These data indicated that basal Aβ uptake in wt microglia is EP2-independent and EP2-mediated suppression of microglial Aβ₁₋₄₂ phagocytosis is largely, if not completely, PKC-dependent.

Basal activation of EP2^{-/-} microglia. As seen in FIG. 1, enhanced Aβ peptide phagocytosis by EP2^{-/-} microglia in human tissue sections was associated with morphological features associated with activation. While this may result from stimulation of microglia by tissue components, it also may result, at least in part, from constitutive activation of EP2^{-/-} microglia; here we investigated the latter (Table 1).

TABLE 1

| | wt microglia (n = 6) | EP2^{-/-} microglia (n = 6) |
|---|---|---|
| Cell number (serum) | 10 ± 1 × 10⁴ | 10 ± 1 × 10⁴ |
| Cell number (serum free) | 8 ± 1 × 10⁴ | 9 ± 1 × 10⁴ |
| MIP-1α (pg/ml) | 1089 ± 49 | 2227 ± 122* |
| MCP-1 (pg/ml) | 633 + 8 | 1112 ± 35* |

Basal activation of EP2^{-/-} microglia. Proliferation of primary cultures of mouse cerebral EP2^{-/-} microglia were compared to wt microglia by counting microglia following 24 hr incubation in medium either containing or free of serum supplement at initial cell number of 1 × 10⁵; no significant difference between microglia from mice of different genotypes was observed (P > 0.05). Cytokine/chemokine array pointed to MIP-1α and
MCP-1 as detectably increased in conditioned medium from primary EP2^{-/-} microglia compared to wt; ELISA data for both chemokines are presented. Data are mean + SEM for n = 6 in each condition (*P < 0.001).

One feature of microglial activation is increased proliferation. We investigated this by counting primary microglia following 24 hr in culture after plating under identical conditions and observed no difference between wt and EP2^{-/-} microglia. Another indicator of activation is cytokine and chemokine secretion. Conditioned culture medium was screened for secretion of 32 cytokines and chemokines using a membrane array that has the advantage of simultaneously measuring multiple molecules but the disadvantage of relative insensitivity. Only two chemokines, MCP-1 and MIP-1α, were sufficiently elevated in conditioned medium from EP2^{-/-} microglial cultures to be detected by cytokine array, while wt microglia had no detectable cytokines or chemokines when simultaneously assayed (data not shown). Data from ELISA confirmed that MCP-1 and MIP-1α were significantly greater in EP2^{-/-} than wt conditioned medium (P<0.001) (Table 1). Macrophage colony stimulating factor (M-CSF), reported to increase microglial phagocytosis of Aβ (Mitrasinovic et al., Neurobiol Aging 24: 807-815, 2003) was not detected by our membrane array or by ELISA (limit of detection of 5 pg/ml) of conditioned medium from wt or EP2^{-/-} microglia. Thus, even without specific exogenous stimuli, EP2^{-/-} microglia displayed some features of an activated phenotype with increased secretion of MCP-1 and MIP-1αcompared to wt microglia, but not increased proliferation by cell count. Moreover, these data indicate that at least some chemoattractant chemokines, viz., MCP-1 and MIP-1α, are regulated by EP2 signaling.

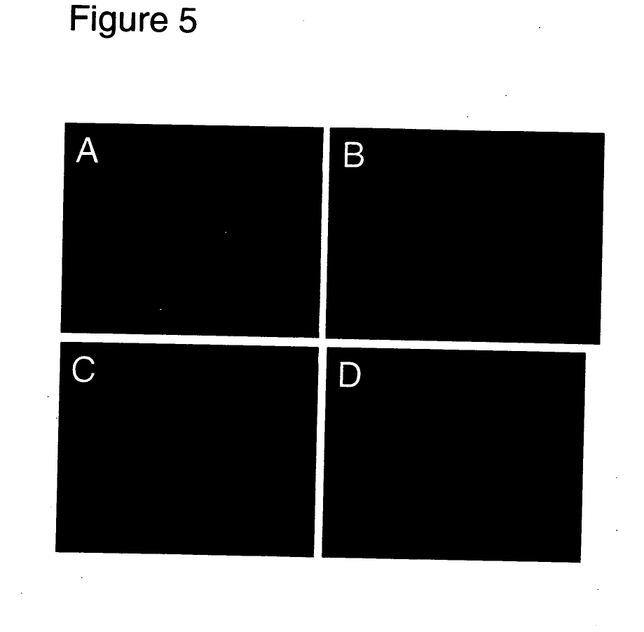

Neuroprotection from Aβ toxicity by EP2^{-/-} microglia. Although enhanced Aβ₁₋₄₂ phagocytosis and reduction of Aβ peptide burden in AD tissue seen with EP2^{-/-} microglia is desirable, if it comes at the cost of increased activation with increased neurotoxicity, then it is not clear if this is a true benefit to patients with AD. To test whether Aβ-activated EP2^{-/-} microglia had enhanced paracrine neurotoxicity, we used primary cultures of wt neurons and microglia from either wt or EP2^{-/-} mice. The final DMSO concentration in these experiments was 0.1% and was non-toxic to primary microglia or neurons. As expected from the results of others, (Qin et al., J. Neurochem. 83: 973-983, 2002; Minagar et al., J. Neurol. Sci. 202: 13-23, 2002; Novarino et al., J. Neurosci. 24: 5322-5330, 2004) exposure of neurons alone to aggregated Aβ₁₋₄₂ was associated with dendrite atrophy (FIG. 5B) and decreased neuron number as assessed by MAP-2 IR (Table 2).

TABLE 2

| | | | |
|---|---|---|---|
| | | MAP-2-IR cells | TUNEL & MAP-2-IR cells |
| Neurons | Microglia | (%; n = 15 each) | (%; n = 15 each) |

| | | | |
|---|---|---|---|
| wt | none | 67 ± 5* | 196 ± 11* |
| wt | wt | 56 ± 4*, \hat{ } | 259 ± 12*, \hat{ } |
| wt | EP2^{-/-} | 102 ± 4 | 59 ± 5⁺ |

Neuroprotection from aggregated Aβ₁₋₄₂ by EP2^{-/-} microglia. Primary neuronal cultures (DIV 6) with or without primary microglia (DIV 14) were exposed to 12 μM of aggregated Aβ₁₋₄₂ for 24 hr. Cultures were then fixed and the total number of MAP-2-IR cells and the remaining number of MAP-2 IR cells that were TUNEL positive were determined. There was no difference among the number of MAP-2 IR cells in neurons only, neuron
plus wt microglia, and neuron plus EP2^{-/-} microglia cultures when exposed to vehicle rather than Aβ₁₋₄₂. Data were presented as % of Aβ₁₋₄₂ -nonexposed neuron only cultures. *P < 0.001 compared to corresponding culture not treated with Aβ₁₋₄₂. ⁺P < 0.05 compared to corresponding culture not treated with Aβ₁₋₄₂. \hat{ }P < 0.05 compared to neuron-only cultures exposed to Aβ₁₋₄₂.

Even greater Aβ₁₋₄₂-initiated dendrite atrophy and neuron loss was observed in cultures that combined neurons and wt microglia (FIG. 5C and Table 2). These results were confirmed in the remaining MAP-2 IR cells that showed increased TUNEL positivity following exposure to Aβ₁₋₄₂ and still further increased TUNEL positivity in MAP-2 IR cells in cultures containing neurons and wt microglia (Table 2). Strikingly, cultures that contained neurons and EP2^{-/-} microglia showed no evidence of neurotoxicity by either of these endpoints or morphologic assessment (Table 2 and FIG. 5D). Indeed, by one measure, TUNEL positivity, co-culture with EP2^{-/-} microglia was associated with apparent neurotrophism. The lack of Aβ₁₋₄₂-mediated neurotoxicity in EP2^{-/-} microglia/wt neuron co-cultures strongly suggests that, while Aβ₁₋₄₂ can be directly toxic to isolated neurons in culture, when combined with microglia the major neurotoxins are paracrine factors secreted by Aβ₁₋₄₂-activated microglia. Wt microglial activation by Aβ yielded the expected large increase in COX-2 and iNOS as determined by Western blot; primary and secondary products of both enzymes can cause neurotoxicity (Manabe et al., Ann Neurol. 55: 668-675, 2004; Andreasson et al., J. Neurosci. 21: 8198-8209; 2001; Dawson et al., Prog Brain Res. 118: 215-229, 1998). Activation of EP2^{-/-} microglia under identical conditions resulted in COX-2 and iNOS induction that were 16±1% and 43±4%, respectively, of wt microglia (n=3 for each, P<0.01 for both). Although the mechanisms by which microglia may damage neurons are more complex than simply COX-2 and iNOS products, these data indicate at least two mechanisms by which AP-activated EP2^{-/-} microglia may be less neurotoxic.

Discussion

Microglia have been proposed to play both neuroprotective and neurotoxic roles in AD pathogenesis. On one hand, phagocytosis of Aβ peptides by activated microglia is thought to be neuroprotective by limiting exposure of neurons to toxic forms of these proteins. On the other hand, exposure of microglia to Aβ peptides initiates an innate immune response with secretion of several neurotoxic factors. Which of these processes is dominant at different points in AD pathogenesis remains obscure; however, it is clear that identifying therapeutic targets that enhance microglial Aβ phagocytosis while also suppressing microglial-mediated neurotoxicity would be a major advance. Our data show that microglial EP2 is such a therapeutic target and demonstrate that it is possible to separate microglial activation with its associated neurotoxicity from enhanced Aβ phagocytosis.

As a justification for Aβ vaccination trials, others have shown that culturing wt rodent or human microglia on human AD brain sections that had been incubated with anti-Aβ antibodies results in clearance of Aβ from tissue and appearance of Aβ-IR material in intracellular phagosomes. Presumably this anti-Aβ antibody-enhanced microglial phagocytosis of tissue Aβ is due to opsonization and Fc receptor-dependent phagocytosis in ex vivo systems (Solomon et al., Proc Natl Acad Sci USA 94: 4109-4112, 1997; Schenk et al., Nature 400: 173-177, 1999; Bard et al., Nat Med 6: 916-919, 2000) although this has been challenged as the mechanism of action of Aβ vaccination in vivo where non-Fc receptor dependent phagocytosis is also involved. (Das et al., J Neurosci. 23: 8532-8538; 2003; Bacskai et al., J Neurosci. 22: 7873-7878, 2002). It is important to note that without incubation of tissue with anti-Aβ antibodies and presumed opsonization of tissue Aβ, wt microglia do not phagocytose Aβ or clear Aβ from human tissue sections ex vivo (Schenk et al., Nature 400: 173-177, 1999). To our knowledge, ours is the first demonstration of genetically altered microglia achieving similar Aβ clearance from human tissue sections without involvement of antibody binding as anti-Aβ opsonization followed by wt microglia reported by others.

Recently, others have shown that EP2 on rodent alveolar macrophages suppresses Fc receptor-mediated phagocytosis of bacterial pathogens (Aronoff et al., J Immunol. 173: 559-565, 2004). Importantly, EP2-mediated suppression of Fc receptor-mediated phagocytosis by alveolar macrophages was cAMP-dependent and the suppressed phagocytic phenotype was restored in EP2^{-/-} macrophages by treatment with forskolin; the participation of EP2 in Fc receptor-independent macrophage phagocytosis is not yet reported. Similar to these investigators, we also observed an EP2-dependent suppressive effect on microglial Aβ phagocytosis; however, this was dependent on PKC activation and was not modified by drugs that activate or inhibit adenylate cyclase. Importantly, we observed that inhibition of PKC in EP2^{-/-} microglia restored the suppressed phagocytic phenotype in EP2⁴^{-/-} microglia to wt levels, strongly suggesting that post-receptor signaling cascades were intact in EP2^{-/-} microglia. Since Aβ phagocytosis is thought to be, at least in part, opsonin-independent and mediated by a group of receptors including formyl peptide receptor, scavenger receptors, and receptor for advanced glycation end-products (Rogers et al., Glia 40: 260-269, 2002), these data raise the possibility that, in contrast to Fc receptor-dependent phagocytosis, EP2-mediated suppression of Fc receptor-independent phagocytosis may be mediated by PKC dependent mechanisms. The exact effectors responsible for this novel mechanism underlying negative regulation of Aβ phagocytosis through EP2 signaling are not entirely clear; however, the mechanism is likely related, at least in part, to increased secretion of pro-phagocytic β chemokines, such as MIP-1α and MCP-1. Since β chemokine signaling during inflammation requires PKC activity (Zhang et al., J Biol. Chem. 278: 12729-12736, 2003; Carnevale et al., J Biol. Chem. 278: 25317-25322; 2003), one possibility is that downstream effectors of EP2 activation suppress phagocytosis by inhibiting β chemokine signaling, activity of PKC isoforms, or both.

If enhanced Aβ phagocytosis resulted from generalized activation of microglia with correspondingly enhanced innate immune response and increased bystander damage to neurons, then the utility of microglial EP2 as a therapeutic target would be unclear. Previously, we have observed in a murine model of activated cerebral innate immune response from intracerebroventricular injection of lipopolysaccharide that neuronal oxidative damage is completely suppressed in EP2^{-/-} mice (Montine et al., J Neurochem. 83: 463-470, 2002); however, interpretation of these data is confounded by microglial and neuronal expression of EP2. Others have shown that peroxisome proliferator-activated receptor γ (PPARγ) agonists can suppress EP2 expression in lung carcinoma cell lines (Combs et al., J Neurosci. 20: 558-567, 2000) and that PPARγ agonists also suppress Aβ-stimulated microglial paracrine neurotoxicity (Han et al., Biochem. Biophys. Res. Commun. 314: 1093-1099, 2004). Therefore, EP2 may be critical in microglia-mediated neurotoxicity. Here we specifically determined that ablation of microglial EP2 only, without any genetic alteration to neurons, completely suppressed Aβ₁₋₄₂ mediated neurotoxicity. Moreover, as suggested by results from TUNEL assay, EP2^{-/-} microglia may even be mildly neurotrophic. Thus, EP2^{-/-} microglia possess the highly desirable complementary traits of enhanced Aβ phagocytosis without enhanced bystander damage to neurons. Given the complications of Aβ vaccination (McGeer et al., Neurobiol. Aging 24: 391-395, 2003), these results point to suppression of microglial EP2 as a perhaps safer but similarly efficacious means of reducing Aβ burden in human brain.

We showed that ablation of microglial EP2 achieved the highly desirable combination of enhancing Aβ phagocytosis while at the same time completely suppressing bystander damage to neurons.

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for preventing or treating a neurological disease in a mammalian subject comprising administering to the mammalian subject a compound capable of inhibiting prostaglandin E2 (PGE2) receptor, wherein the compound is administered in an amount effective to treat the neurological disease or to prevent its occurrence or recurrence.

2. The method of claim 1 wherein the PGE2 receptor is subtype EP2.

3. The method of claim 1 wherein the inhibitor is a small chemical compound, short interfering RNA, dominant-negative molecule, short hairpin RNA, ribozyme, antisense oligonucleotide, antibody, peptide or peptidomimetic.

4. The method of claim 1 wherein the neurological disease is Alzheimer's disease or Parkinson's disease.

5. The method of claim 1 wherein the compound promotes β amyloid (Aβ) clearance.

6. The method of claim 1 wherein the compound enhances microglial phagocytosis of pathogenic proteins associated with neurological diseases and disorders.

7. The method of claim 6 wherein the pathogenic proteins are Aβ proteins associated with the initiation and progression of Alzheimer's disease.

8. The method of claim 6 wherein the pathogenic proteins are α-synuclein proteins associated with the initiation and progression of Parkinson's disease.

9. An in vitro method of screening for an inhibitor of PGE2 receptor biological activity comprising: contacting a cell with a test compound; and detecting an increase or a decrease in activation, thereby identifying the test compound as an inhibitor of EP receptor biological activity.

10. The method of claim 9 wherein the PGE2 receptor is EP2.

11. The method of claim 9 wherein the cell is an astrocyte, microglia or neuron.

12. A compound identified by the method of claim 9.

13. A method for identifying a compound capable of inhibiting PGE2 receptor signaling comprising:
contacting a test compound with a cell-based assay system comprising a cell expressing a PGE2 receptor capable of signaling responsiveness to PGE2 receptor signaling, and
detecting an effect of the test compound on PGE2 receptor signaling in the assay system as an increase or a decrease in second messenger signaling or physiologic outcome such as increase or decrease in phagocytosis, effectiveness of the test compound in the assay being indicative of the inhibition EP receptor signaling.

14. The method of claim 13, wherein the physiologic outcome is an increase or decrease in phagocytosis.

15. The method of claim 13, wherein the test compound is a small chemical molecule, interfering RNA, short hairpin RNA, ribozyme, antisense oligonucleotide, protein inhibitor, monoclonal antibody, a polyclonal antibody, a peptide, or a nucleic acid.

16. The method of claim 13 wherein the PGE2 receptor is subtype EP2.

17. A compound identified according to the method of claim 15.

18. The compound of claim 17 wherein the compound is an E2 receptor subtype antagonist.

19. The compound of claim 17 wherein the compound alters the biological activity of the EP2 receptor subtype.

20. The compound of claim 17 wherein the compound is an EP2 receptor subtype antagonist.

21. The compound of claim 17 wherein the compound alters the biological activity of the EP2 receptor subtype.

22. A pharmaceutical composition comprising an PGE2 inhibitor for the treatment of a neurological disease or disorder in a mammalian subject.

23. The composition of claim 22 wherein the PGE2 is subtype EP2.

24. The composition of claim 22 wherein the inhibitor is a small chemical compound, interfering RNA, dominant-negative molecule, short hairpin RNA, ribozyme, antisense oligonucleotide, or protein inhibitor.

25. The composition of claim 22 wherein the neurological disease is Alzheimer's disease or Parkinson's disease.

26. The composition of claim 22 wherein the composition promotes Aβ clearance from the brain.

27. The composition of claim 22 wherein the composition promotes synuclein clearance from the brain.

28. The composition of claim 27 wherein synuclein is α-synuclein.
